(19) United States
(12) Patent Application Publication
(10) Pub. No.: US 2015/0114396 A1
(43) Pub. Date: Apr. 30, 2015

(54) METHODS AND APPARATUS FOR PRESSURE TREATMENT MODULATION

(71) Applicant: ResMed Limited, Bella Vista (AU)

(72) Inventors: Dinesh Ramanan, Sydney (AU); Jeffrey Peter Armitstead, Sydney (AU)

(73) Assignee: ResMed Limited, Bella Vista (AU)

(21) Appl. No.: 14/398,266

(22) PCT Filed: May 1, 2013
(86) PCT No.: PCT/AU2013/000448

§ 371 Date: Oct. 31, 2014

Related U.S. Application Data
(60) Provisional application No. 61/641,504, filed on May 2, 2012.

(51) Int. Cl. A61M 16/00; A61M 16/20; A61M 16/06

(52) U.S. Cl. CPC A61M 16/0069; A61M 16/0003; A61M 16/0622; A61M 16/20; A61M 2016/003; A61M 2016/0027; A61M 2205/3334; A61M 2205/52
USPC 128/204.23; 128/204.21

(57) ABSTRACT

Respiratory pressure treatment apparatus include automated methodologies for controlling modulation of pressure during an inspiratory phase or an expiratory phase of patient respiration. The changes in pressure result in various pressure waveforms that may be suitable for treating patients suffering from respiratory insufficiency such as Chronic Obstructive Pulmonary Disease. In example embodiments, a pressure rise or pressure increase may be controlled during a period of patient expiration by implementation of linear, cubic and/or quartic functions that serve as control parameters in a processor that controls a flow generator. One or more of the functions may optionally serve as a control parameter to control the pressure increase during an expiration period and a following decrease during the period of expiration. In some embodiments, such functions may further control a decrease in pressure during a period of patient inspiration, such as a decrease prior to mid-inspiration.

### 1 CROSS-REFERENCE TO RELATED APPLICATIONS

This application claims the benefit of U.S. provisional patent application No. 61/641,504 filed May 2, 2012, the disclosure of which is incorporated herein in its entirety by reference.

### 1.1 FIELD OF THE TECHNOLOGY

The present technology relates to methods and apparatus for controlling respiratory treatment apparatus such as for the treatment of respiratory insufficiency. More particularly, certain embodiments relate to methods and apparatus for pressure control in the treatment of respiratory insufficiency such as Chronic Obstructive Pulmonary Disease.

### 1.2 DESCRIPTION OF THE RELATED ART

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the air into the venous blood and carbon dioxide to move out. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See West, Respiratory Physiology—the essentials.

A range of respiratory disorders exist.

Obstructive Sleep Apnea (OSA), a form of Sleep Disordered Breathing (SDB), is characterized by occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is a disorder of a patient's respiratory controller in which there are rhythmic alternating periods of waxing and waning ventilation, causing repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

Otherwise healthy individuals may take advantage of systems and devices to prevent respiratory disorders from arising.

The diagnosis of CSR usually involves conducting a sleep study and analyzing the resulting polysomnography (“PSG”) data. In a full diagnostic PSG study, a range of biological parameters are monitored that typically include a nasal flow signal, measures of respiratory effort, pulse oximetry, sleeping position, and may include: electroencephalography (“EEG”), electrocardiography (“ECG”), electromyography (“EMG”) and electro-oculography (“EOG”). Breathing characteristics are also identified from visual features, thus allowing a clinician to assess respiratory function during sleep and evaluate any presence of CSR. While the examination by a clinician is the most comprehensive method, it is a costly process and depends heavily upon clinical experience and understanding.

Respiratory insufficiency affects millions of people. For patients suffering from this condition, the lungs are unable to inspire sufficient oxygen or expel sufficient carbon dioxide to meet the needs of the cells of the patient's body. For example, Chronic Obstructive Pulmonary Disease (“COPD”) affects approximately thirteen million Americans and ten million Europeans. COPD is a disease involving some damage to the lungs. The walls of the airways and the alveoli of the lungs can lose their elastic quality. Walls between alveoli can become destroyed or they can become inflamed. The walls of the airways within the lungs may also produce more mucus than usual, which can restrict airflow. This damage will typically manifest itself in some difficulty with breathing such as dyspnea. COPD patients typically experience coughing, with an expulsion of mucus, shortness of breath, wheezing and a feeling of tightness in the chest. Emphysema and chronic obstructive bronchitis may each be considered to be a form of COPD. Chronic obstructive bronchitis may be characterized by an inflammatory response in the larger airways of the lungs. Emphysema may be characterized by destruction of tissue of the lungs from an inflammatory response. Such changes in the lung tissue may result in well-known problems, such as Dynamic pulmonary Hyperinflation (DH) and intrinsic positive end-expiratory pressure (PEEPi).

Cigarette smoking is considered a leading cause of COPD. Most people with COPD have some history of smoking. Extensive exposure to lung irritants, such as air pollution or chemical fumes, may also contribute to COPD.

COPD may develop slowly, with the symptoms worsening over time. COPD is a significant cause of disability. COPD can make it difficult to perform physical activities such as walking or exercise. Initially, the symptoms may be most evident during vigorous activities. However, as the disease progresses, symptoms may become more evident during milder activities and even while at rest. COPD may even lead to death. It is presently the fourth leading cause of death in the United States.

There is no presently known cure for COPD. There is no treatment for restoring the airways and alveoli of the lungs of a COPD patient to their pre-disease condition. However, treatments and lifestyle changes can help a COPD patient to feel more comfortable, continue to be active and impede the progression of the disease.

It will be appreciated that there is a need in the art for techniques and devices for treating the conditions of patients suffering from respiratory insufficiency or COPD that offer either an improved outcome or a useful alternative to the current state of the art.

### 2 BACKGROUND OF THE INVENTION

### 2.1.1 Therapy

Nasal Continuous Positive Airway Pressure (CPAP) therapy has been used to treat Obstructive Sleep Apnea (OSA). The hypothesis is that continuous positive airway pressure acts as a pneumatic splint and may prevent upper airway obstruction by pushing the soft palate and tongue forward and away from the posterior oropharyngeal wall.

Non-invasive ventilation (NIV) has been used to treat CSR, OHS, COPD, MD and Chest Wall disorders. In some cases of NIV, the pressure treatment may be controlled to enforce a target ventilation by measuring a tidal volume or minute ventilation, for example, and controlling the measure of ventilation to satisfy the target ventilation. Servo-controlling of the measure of ventilation, such as by a comparison of an instantaneous measure of ventilation and a long term measure of ventilation, may serve as a treatment to counteract CSR. In some such cases, the form of the pressure treatment delivered by an apparatus may be Pressure Support ventilation. Such a pressure treatment typically provides generation of a higher level of pressure during inspiration (e.g., an IPAP) and generation of a lower level of pressure during expiration (e.g., an EPAP).

### 2.1.2 Patient Interface

The application of a supply of air at positive pressure to the entrance of the airways of a patient is facilitated by the use of a patient interface, such as a nasal mask, full-face mask or nasal pillows. A range of patient interface devices are known, however a number of them suffer from being one or more of obtrusive, aesthetically undesirable, poorly fitting, difficult to use and uncomfortable especially when worn for long periods of time or when a patient is unfamiliar with a system. Masks designed solely for aviators, as part of personal protection equipment or for the administration of anaesthetics may be tolerable for their original application, but nevertheless be undesirably uncomfortable to be worn for extended periods, for example, while sleeping.

### 2.1.3 Systems

One known product used for treating sleep disordered breathing is the S9 Sleep Therapy System, manufactured by ResMed.

### 3 BRIEF SUMMARY OF THE TECHNOLOGY

An aspect of certain example embodiments of the present technology relates to automated control methodologies for respiratory pressure treatment apparatus implemented to treat respiratory insufficiency.

### 3.1.1 PAP Device

The air at positive pressure is typically supplied to the airway of a patient by a PAP device such as a motor-driven blower. The outlet of the blower is connected via a flexible delivery conduit to a patient interface as described above.

Periodic breathing disorders of central origin, such as Cheyne-Stokes respiration, may occur together with upper airway obstruction. The oscillations in central drive to the respiratory musculature may be associated with oscillations in drive to the upper airway musculature, exacerbating any tendency to upper airway obstruction. Any method which attempts to counteract the self-sustaining oscillations in respiratory drive by ventilating the patient, typically with more ventilator drive during periods of low patient effort than during periods of high patient effort, needs the upper airway to be substantially open when it is attempting to deliver ventilatory assistance, otherwise the ventilatory assistance will be to some extent, and often totally, ineffective during the periods of low or zero patient effort, and thus unable to stabilise the patient's ventilation.

This need to keep the upper airway open is typically addressed by attempting to set an expiratory positive airway pressure (EPAP) such that the upper airway is kept open at all times. This may be achieved by some kind of iterative adjustment of EPAP while observing indicators of the patency of the airway at various EPAP levels, in a procedure called a titration. Titration is a skilled and typically expensive operation, preferably being conducted in a sleep laboratory, and may not yield an EPAP sufficient to overcome upper airway obstruction (UAO). Reasons for this include the fact that UAO is often postural, and the patient may never during the titration night assume the posture which produces the worst UAO, typically the supine posture. Sedative and other drugs may variably influence the upper airway. There is also evidence that the degree of cardiac failure affects the degree of upper airway obstruction via oedema of the upper airway. Hence an exacerbation of cardiac failure may worsen upper airway obstruction to an extent which cannot be anticipated during a titration night.

Some embodiments of the present technology may involve a respiratory pressure treatment apparatus. The apparatus may include a flow generator to generate a flow of breathable gas to a patient interface. The apparatus may also include a sensor to measure the flow of breathable gas. The apparatus may also include a controller to control the flow generator to deliver a flow of breathable gas at a patient interface. The flow of breathable gas may be adjusted as a function of a detection of a respiratory cycle. The flow of breathable gas may comprise inspiratory portions and expiratory portions, wherein an expiratory portion may be at a pressure generally lower than that during an inspiratory portion, and each of the expiratory pressure portions may comprise a pressure rise. The controller may also be configured to control the pressure rise of the expiratory pressure portion with a polynomial function.

Optionally, in some such cases, the polynomial function may comprise a function of time or phase. The polynomial function may be linear. The polynomial function may be a cubic function. The polynomial function may be a quartic function. The polynomial function may comprise a sum of products of a set of coefficients and an input parameter, the input parameter being a measure of at least one of a respiratory flow, a respiratory phase and a respiratory time. The set of coefficients may be selected as a function of detected respiratory phase. Optionally, a first set of coefficients may be selected for an early portion of expiration and a second set of coefficients may be selected for a latter portion of expiration. The controller may be further configured to control a pressure decline in the expiratory portion such that the pressure decline may be subsequent to the pressure rise. The pressure decline may be controlled with the polynomial function. Optionally, the controller may include an intra-expiratory cycling point setting, wherein the controller may be configured to control the pressure decline as a function of the intra-expiratory cycling point setting. The controller may be configured to control an inspiratory portion with a polynomial function of at least a degree of three. The control of the pressure of the inspiratory portion may decrease the pressure during patient inspiration. The decrease of pressure of the inspiratory portion may follow an increase of pressure in the inspiratory portion. The control of the pressure rise may be a further function of a maximum pressure support setting value.

Another aspect of some embodiments the present technology is the automated control of pressure to provide modulated waveforms during portions of patient inspiration and/or portions of patient expiration.

In some embodiments, automated control of various waveforms may be implemented by pressure templates such as a template derived from one or more of a linear, cubic and quartic function.

In some embodiments, automated control of expiratory pressure modulation may be implemented by an intra-expiratory cycling threshold.

In some embodiments, automated control of inspiratory pressure modulation may be implemented with an intra-inspiratory cycling threshold.

Some embodiments of the technology may involve a method of control for a respiratory pressure treatment device. The method may include generating a flow of breathable gas at a patient interface. The flow of breathable gas may be adjusted as a function of a detection of a respiratory cycle. The such that the flow of breathable gas may comprises inspiratory pressure portions and expiratory pressure portions wherein the breathable gas during an expiratory pressure portion is at a pressure generally lower than that during an inspiratory pressure portion and wherein each of the expiratory pressure portions comprises a pressure rise. The method may also include controlling the pressure rise of the expiratory pressure portions with a polynomial function.

Optionally, the polynomial function may comprise a function of time or phase. The polynomial function may be linear. For example, it may be a linear function of elapsed respiratory time. The polynomial function may be a cubic function. The polynomial function may be a quartic function. The polynomial function may comprise a sum of products of a set of coefficients and an input parameter, the input parameter being a measure of at least one of a respiratory flow, a respiratory phase and respiratory time. The set of coefficients may be selected as a function of detected respiratory phase. Optionally, a first set of coefficients may be selected for an early portion of expiration and second set of coefficients may be selected for a latter portion of expiration. In some such embodiments, the method may also include controlling a pressure decline in the expiratory portion, the pressure decline being subsequent to the pressure rise. The pressure decline may be controlled with the polynomial function. The control of the pressure decline may be a function of an intra-expiratory cycling point setting value. In some embodiments, the method may also include controlling an inspiratory pressure portion with a polynomial function of at least a degree of three. Optionally, the control of the pressure of the inspiratory pressure portion may decrease the pressure during patient inspiration. The decrease of pressure of the inspiratory pressure portion may follow an increase of pressure in the inspiratory portion. Optionally, the control of the pressure rise may be a further function of a maximum pressure support setting value.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF SEVERAL VIEWS OF THE DRAWINGS

The present technology is illustrated by way of example, and not by way of limitation, in the figures of the accompanying drawings, in which like reference numerals refer to similar elements including:

### 4.1 TREATMENT SYSTEMS

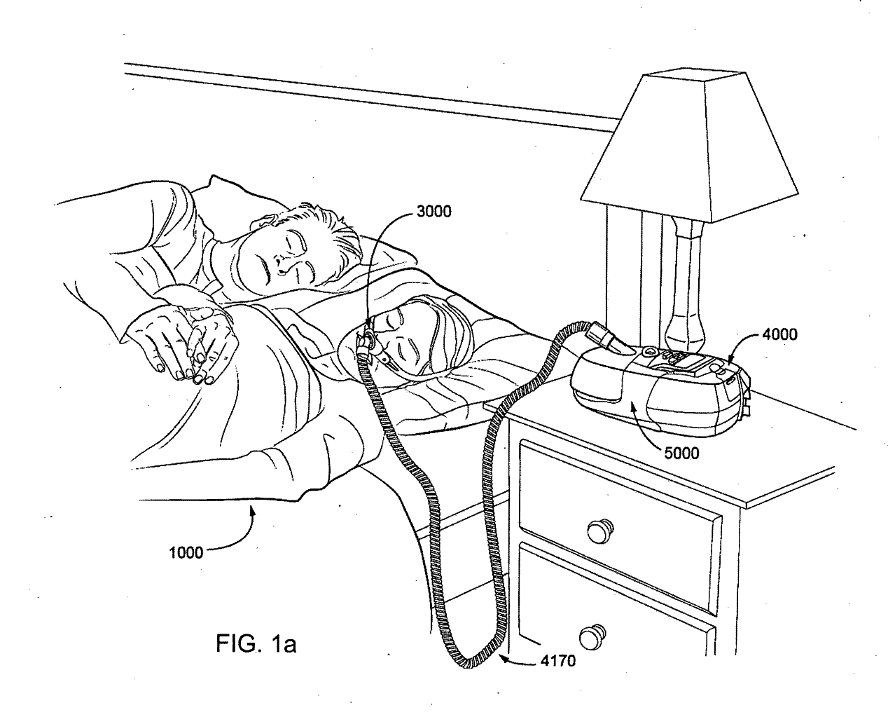

FIG. 1a shows a system relating to the present technology. A patient 1000 wearing a patient interface 3000, receives a supply of air at positive pressure from a PAP device 4000. Air from the PAP device is humidified in a humidifier 5000, and passes along an air circuit 4170 to the patient 1000.

### 4.2 THERAPY

### 4.2.1 Respiratory System

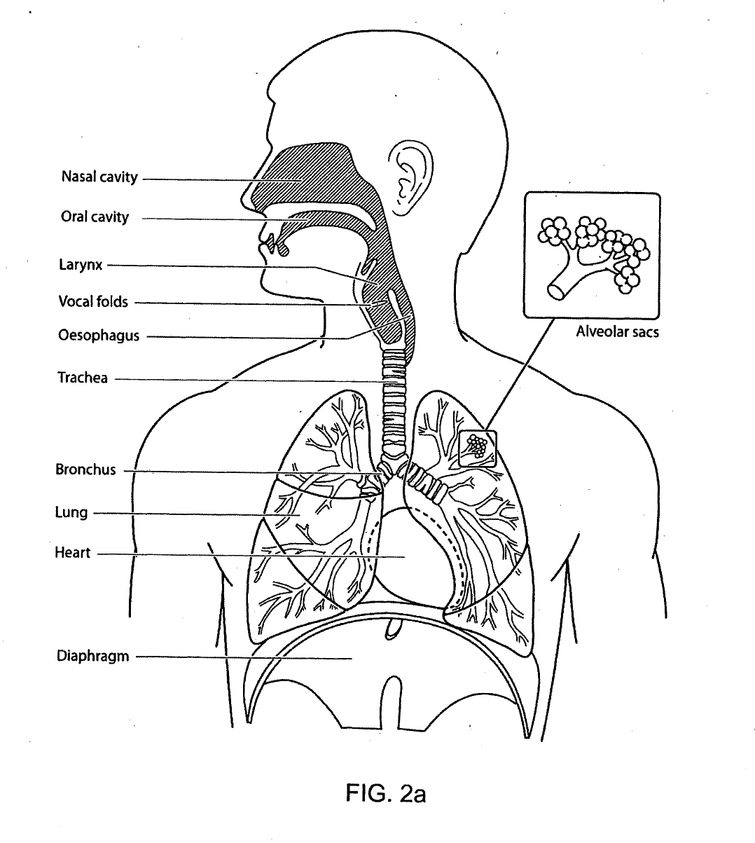

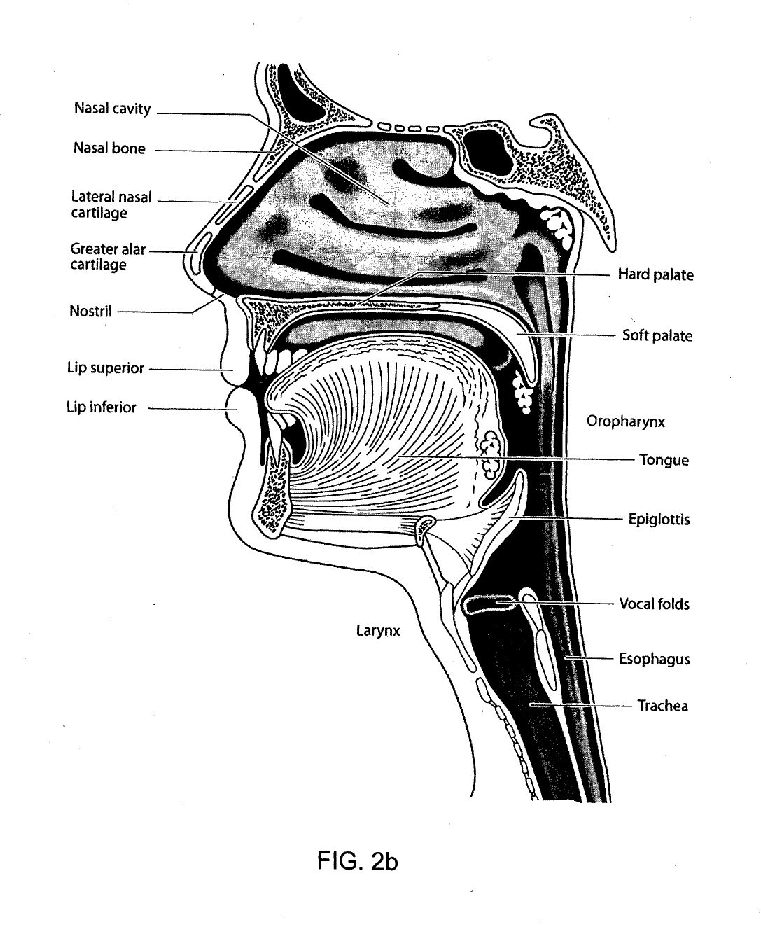

FIG. 2a shows an overview of a human respiratory system including the nasal and oral cavities, the larynx, vocal folds, oesophagus, trachea, bronchus, lung, alveolar sacs, heart and diaphragm.

FIG. 2b shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

### 4.3 PATIENT INTERFACE

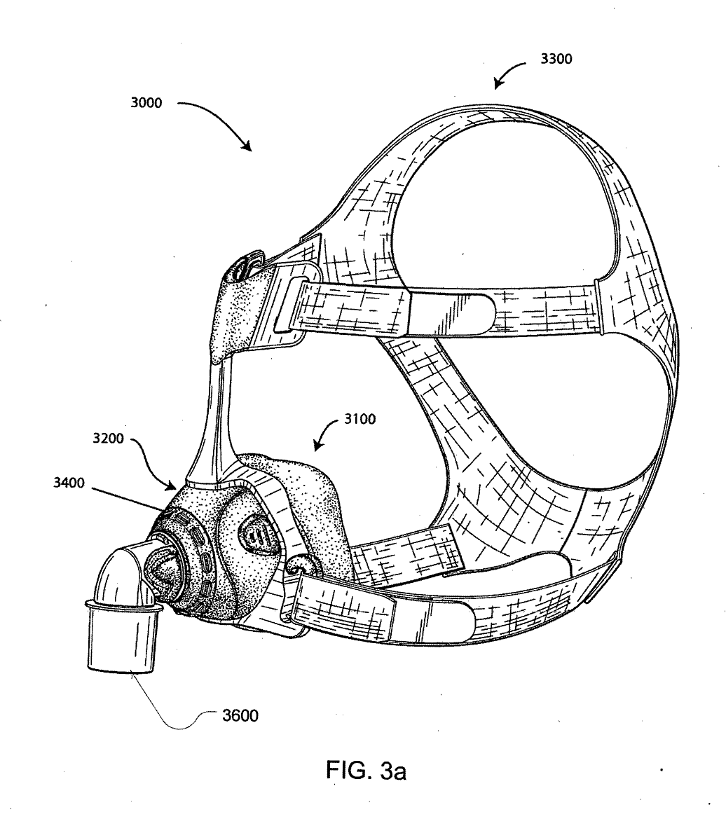

FIG. 3a shows a traditional mask-type patient interface.

### 4.4 PAP DEVICE

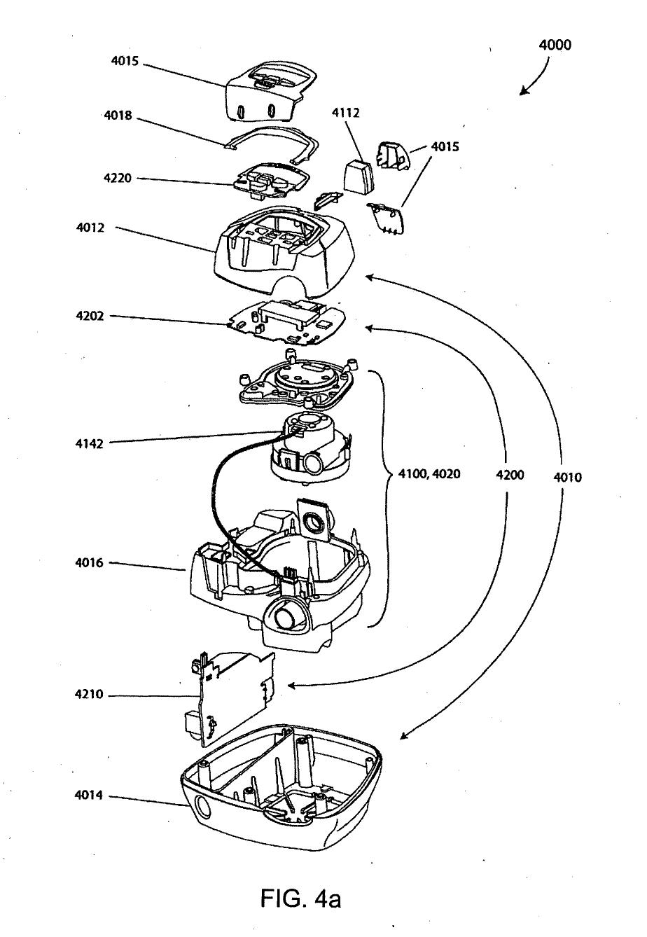

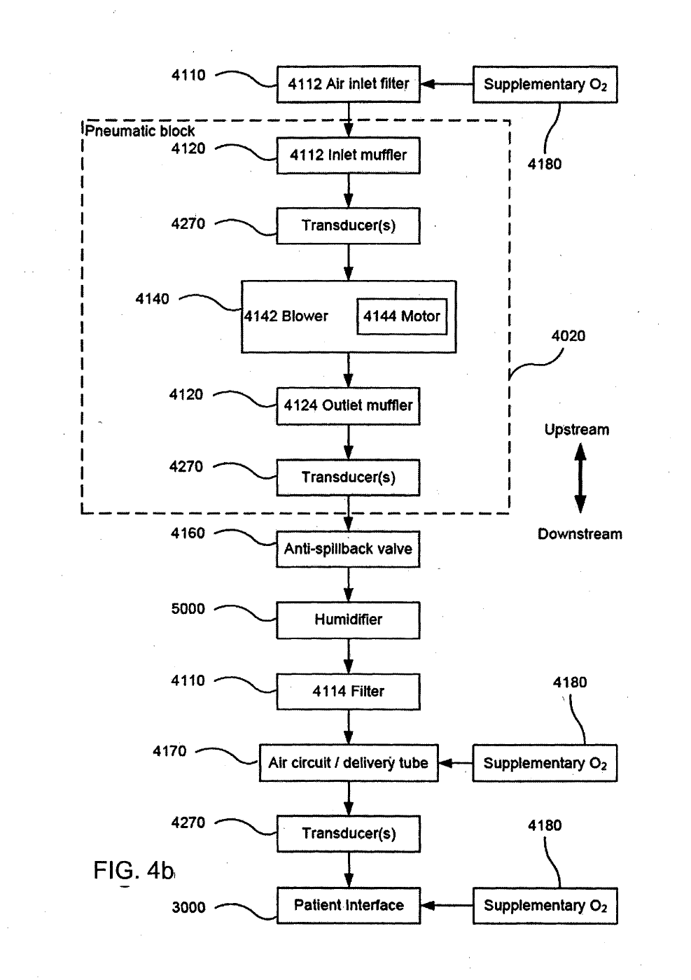

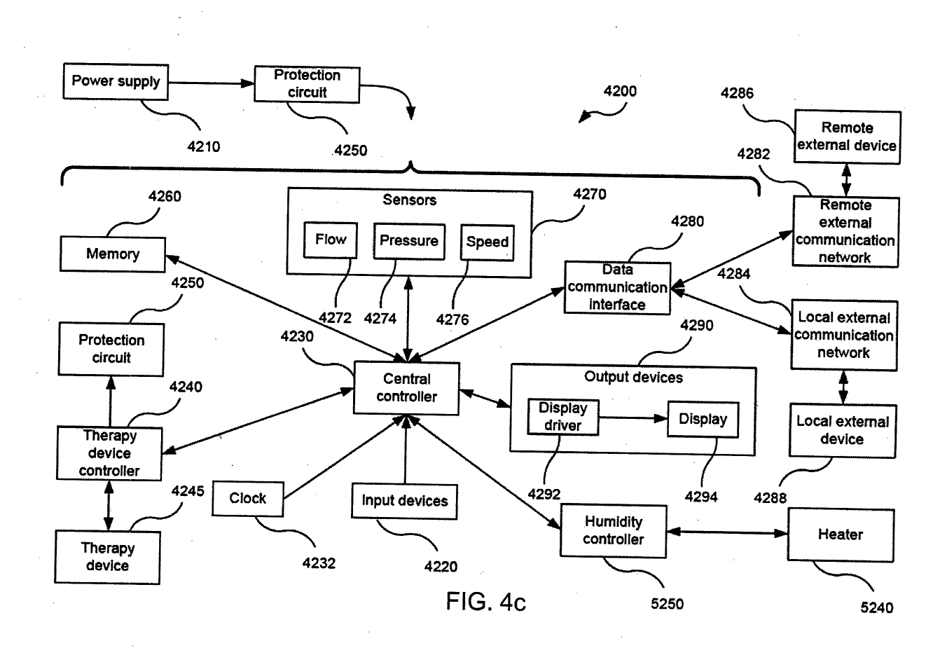

FIG. 4a shows a PAP device in exploded view in accordance with one form of the present technology.

FIG. 4b shows a schematic diagram of the pneumatic circuit of an example PAP device of FIG. 4a. The directions of upstream and downstream are indicated.

FIG. 4c shows a schematic diagram of example electrical components of the PAP device of FIG. 4a.

### 4.5 HUMIDIFIER

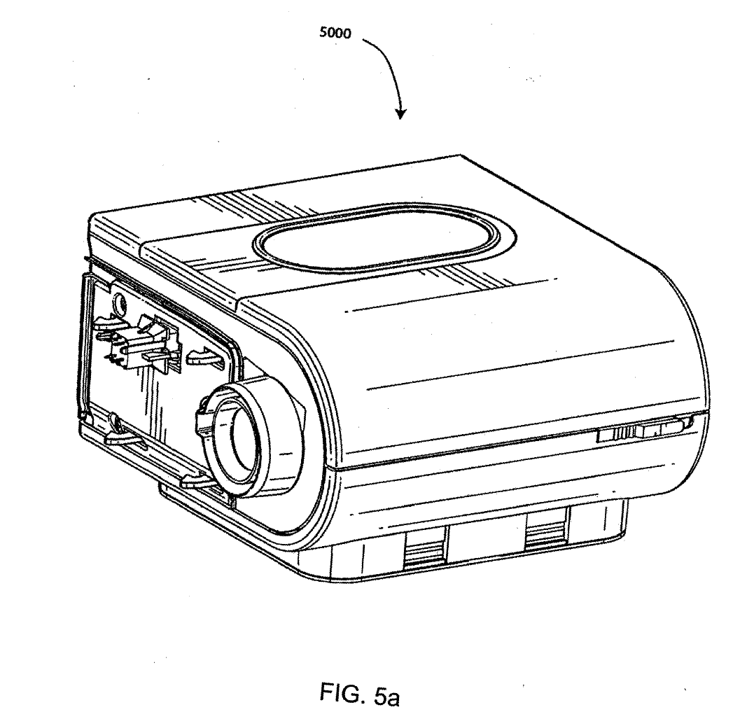

FIG. 5a shows a traditional humidifier in accordance with one aspect of the present technology.

### 4.6 PRESSURE TREATMENT MODULATION SYSTEM

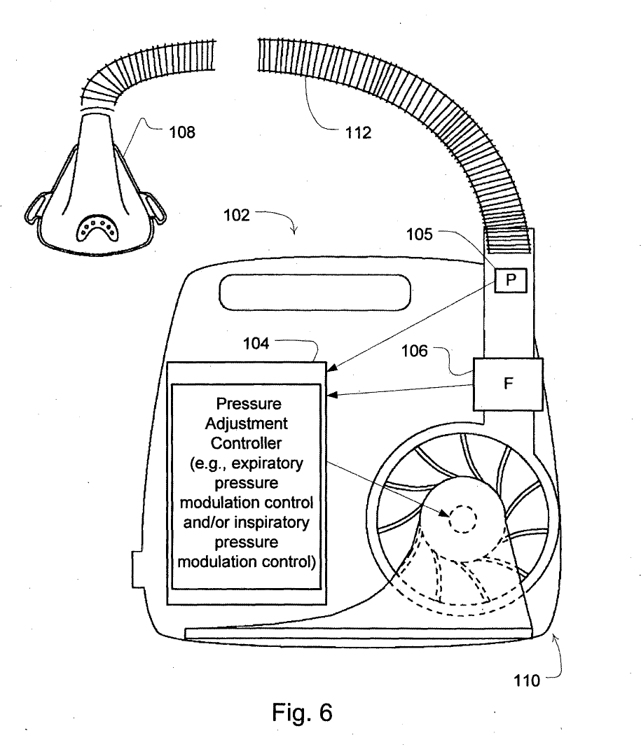

FIG. 6 illustrates example components of a respiratory pressure treatment device of the present technology;

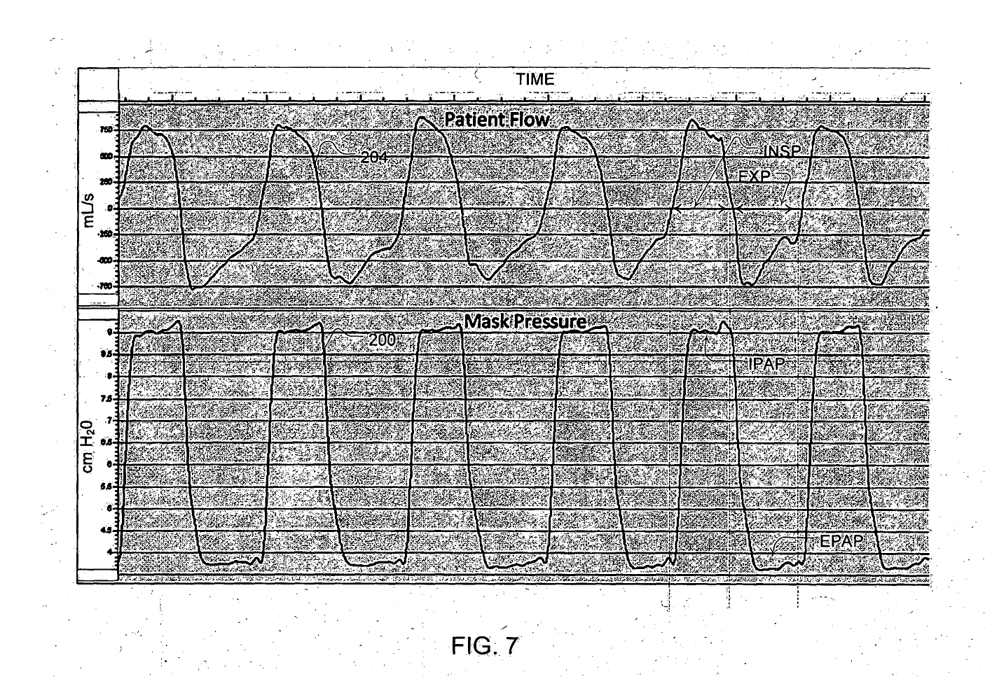

FIG. 7 is a flow and pressure versus time graph illustrating a typical bi-level pressure treatment over several breathing cycles;

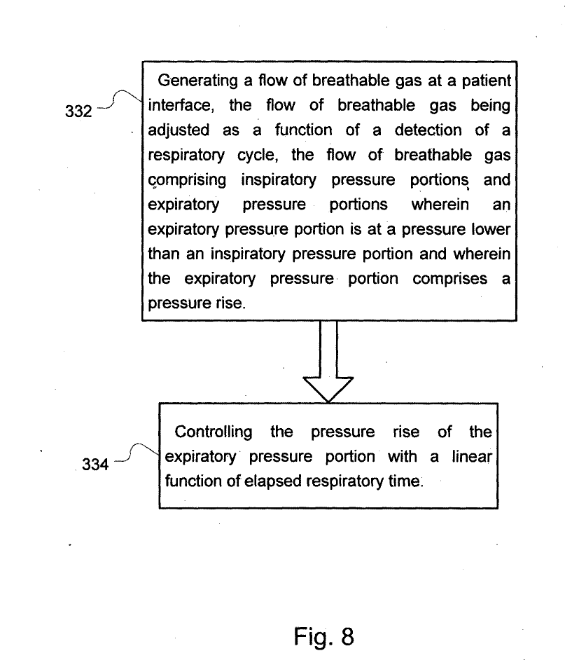

FIG. 8 is an example control methodology for expiratory pressure modulation;

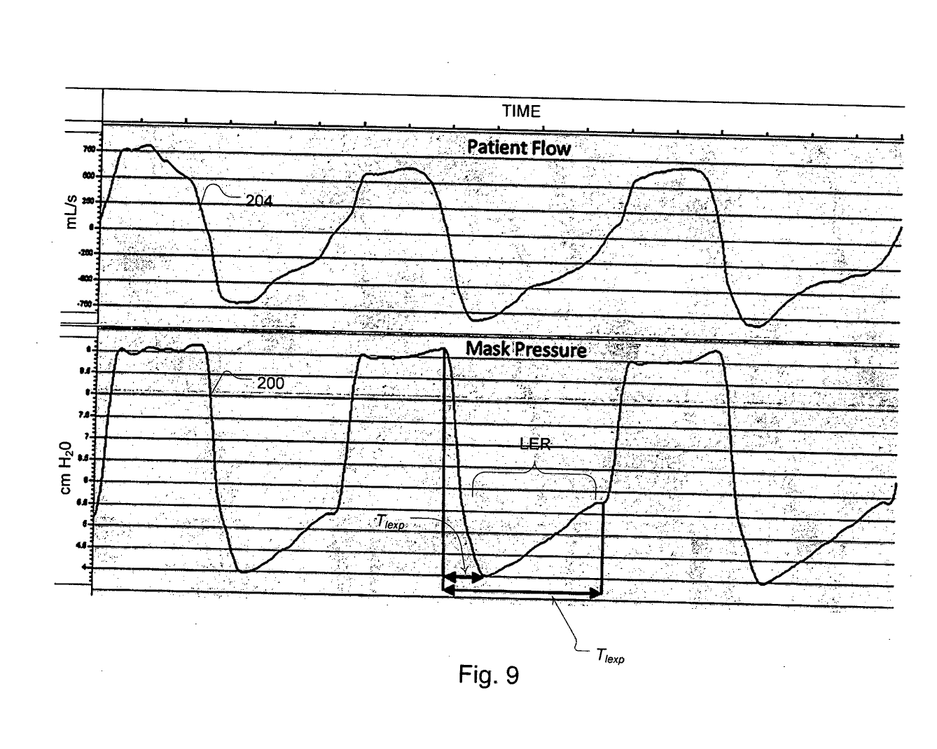

FIG. 9 illustrates an example pressure waveform with linear expiratory modulation made in accordance with the methodology of FIG. 3;

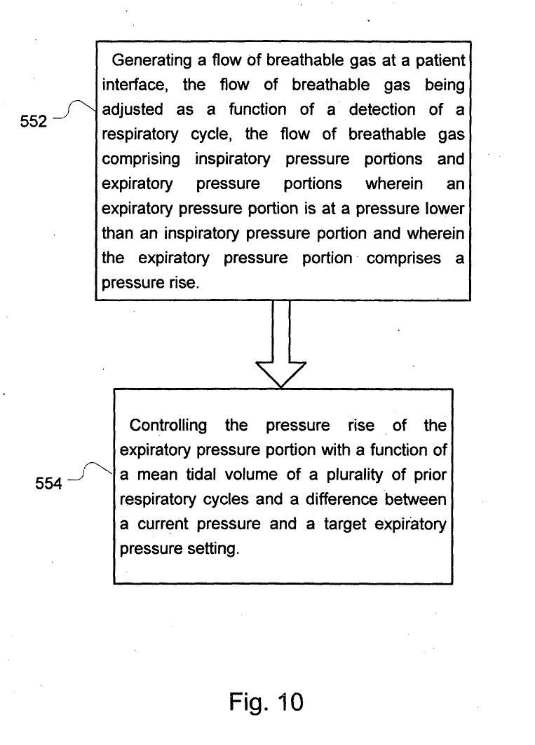

FIG. 10 is an further example control methodology for expiratory pressure modulation;

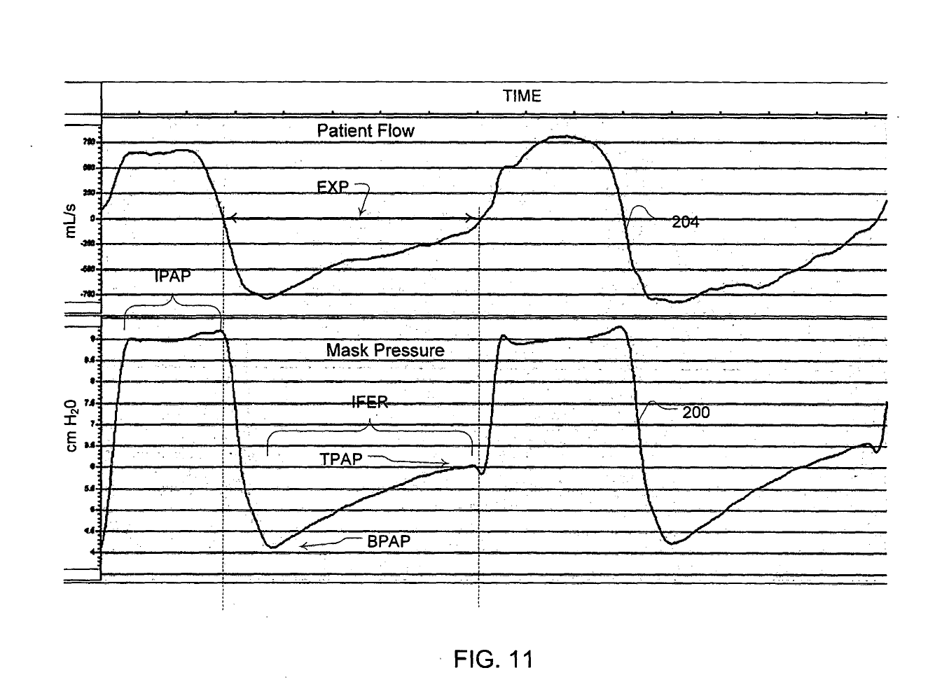

FIG. 11 illustrates an example pressure waveform with tidal volume based expiratory modulation in accordance with the methodology of FIG. 5;

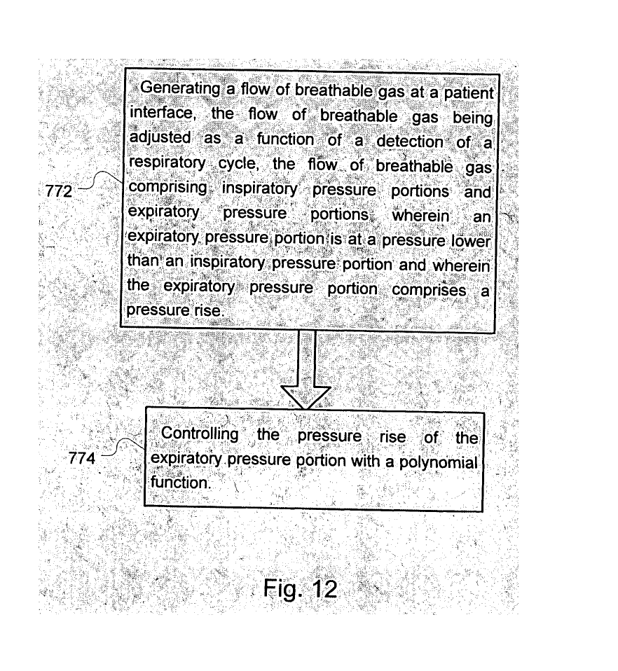

FIG. 12 is a further example control methodology for expiratory pressure modulation of the present technology;

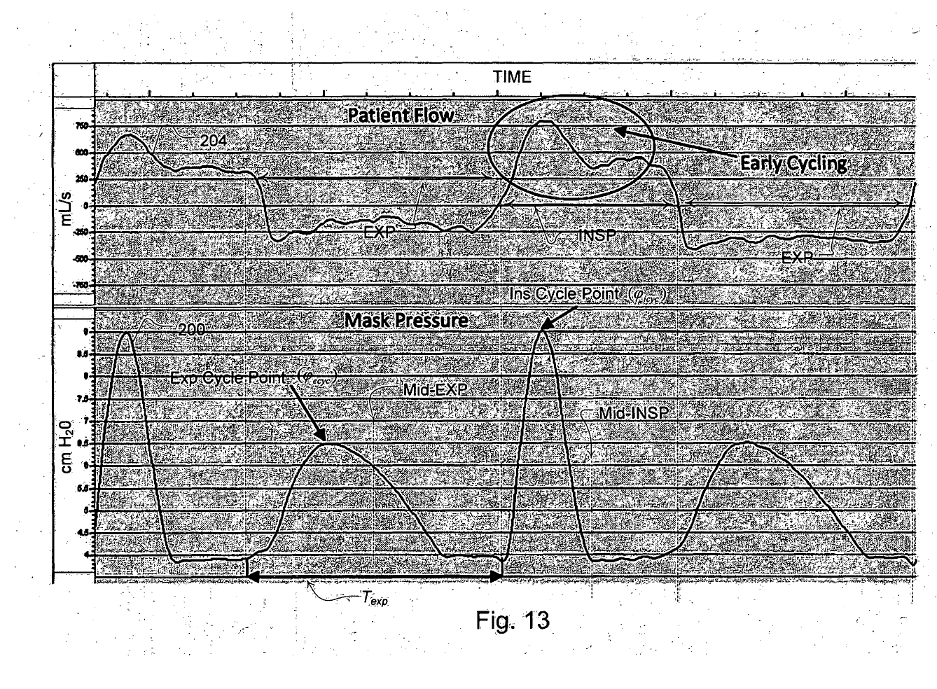

FIG. 13 illustrates example pressure waveforms with cubic function modulations in accordance with the example methodology of FIG. 12;

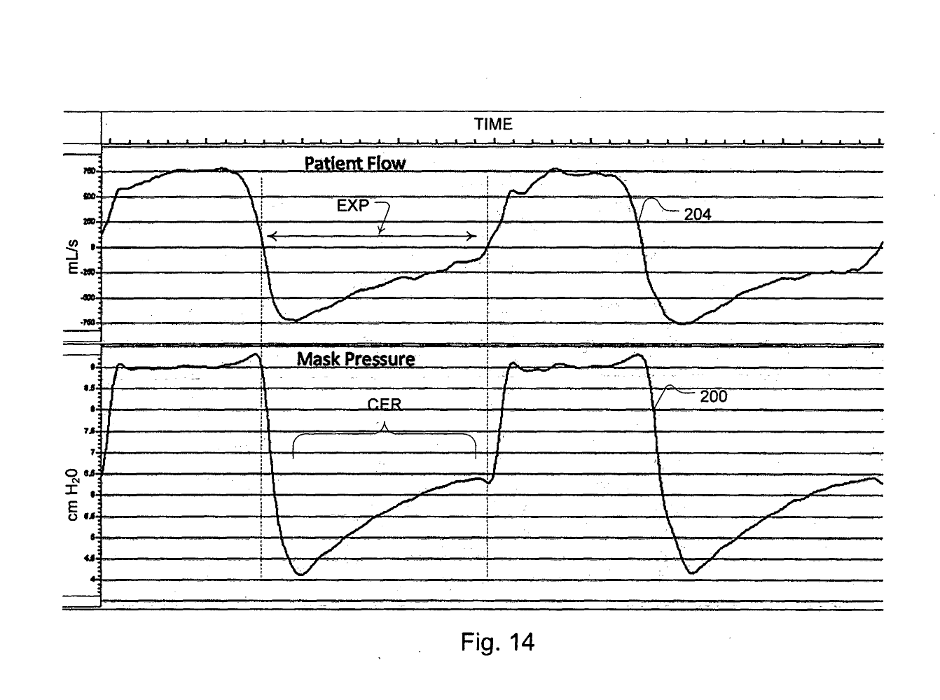

FIG. 14 illustrates further example pressure waveforms with cubic expiratory raise in accordance with the example methodology of FIG. 12;

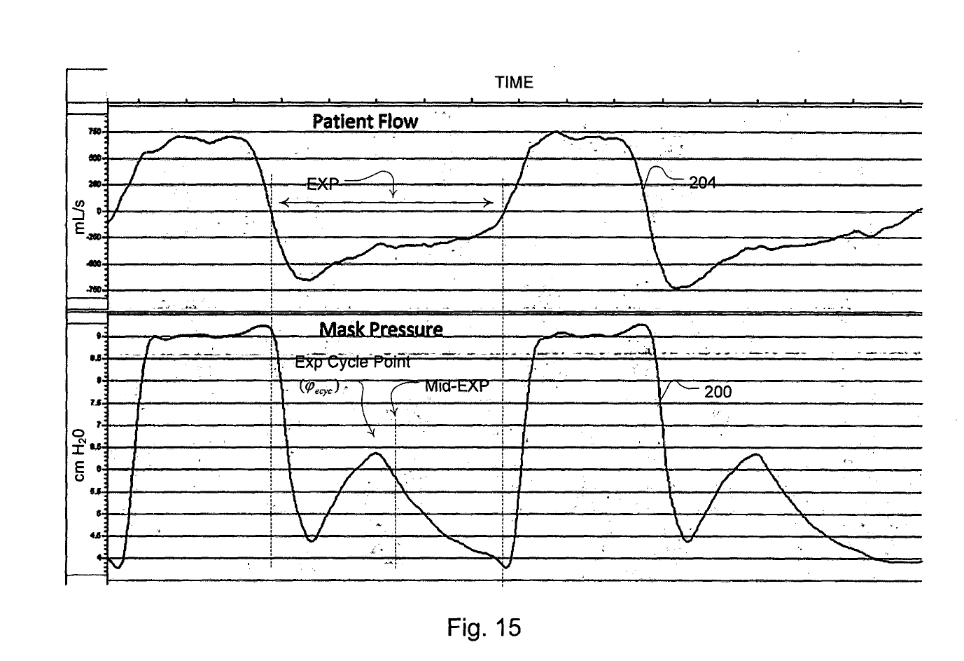

FIG. 15 illustrates still further example pressure waveforms with cubic expiratory modulations in the form of an expiratory raise and fall, in accordance with the example methodology of FIG. 12;

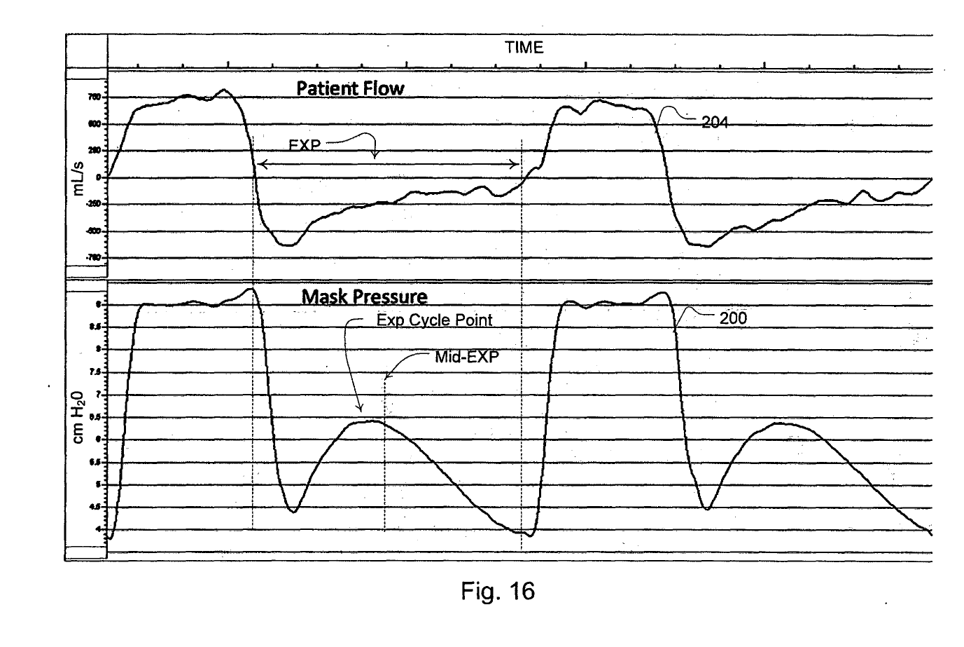

FIG. 16 illustrates still further example pressure waveforms with quartic expiratory modulations in accordance with the example methodology of FIG. 12;

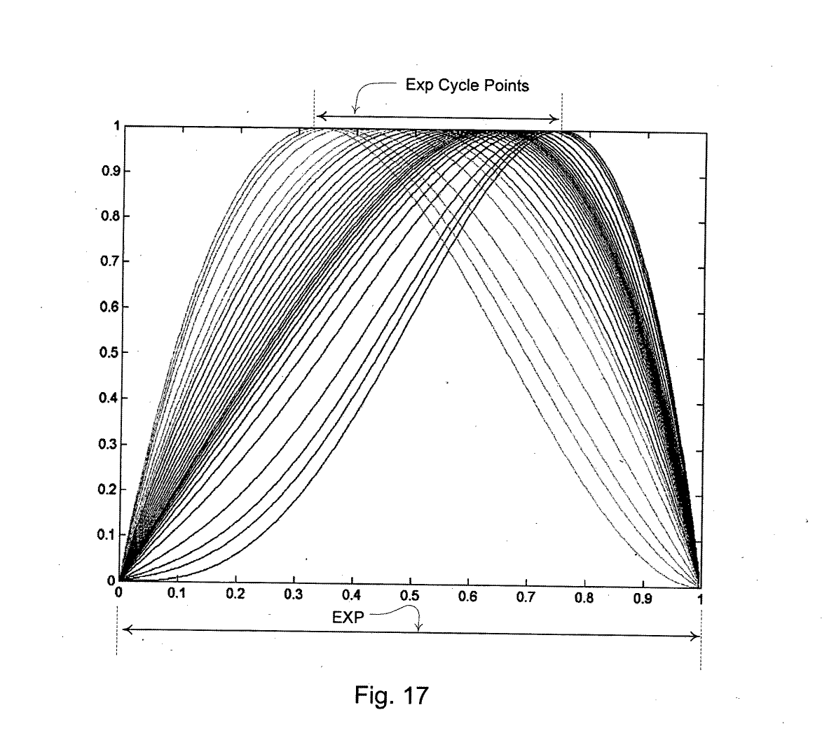

FIG. 17 illustrates still further example pressure templates with quartic expiratory modulation in accordance with the example methodology of FIG. 12; and

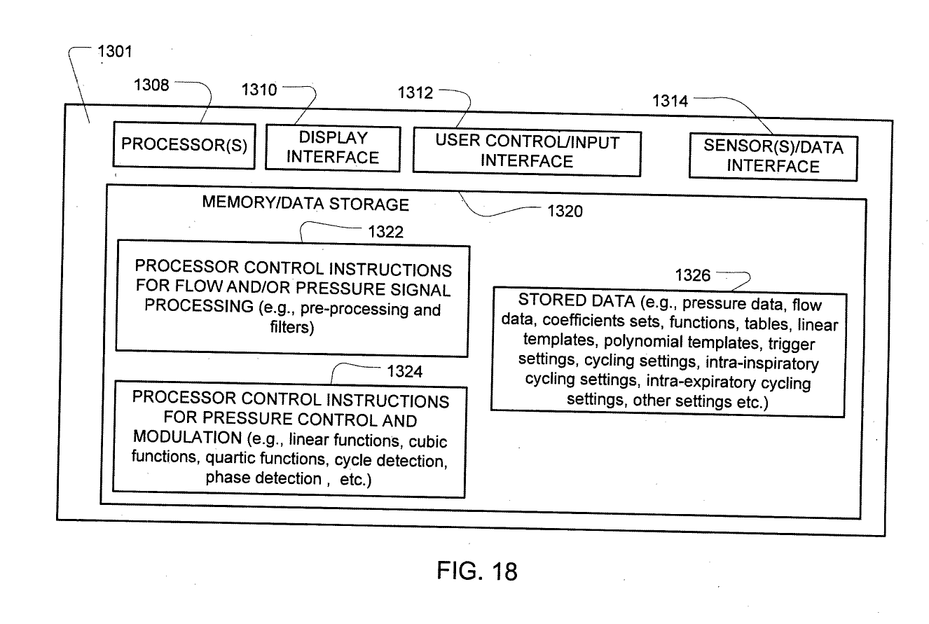

FIG. 18 illustrates a block diagram for an example controller architecture of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

### 5.1 TREATMENT SYSTEMS

In one form shown in FIG. 1, the present technology comprises apparatus for treating a respiratory disorder. The apparatus may comprise a flow generator or blower for supplying pressurised respiratory gas (e.g., PAP device 4000), such as air, to the patient 1000 via an air delivery tube leading to a patient interface 3000.

For example, the present technology can involve methods and devices for the modulation of pressure treatment such as for patients with respiratory insufficiency or COPD. In particular, the present technology attempts to mitigate the problem of intrinsic positive end expiratory pressure (PEEPi) by applying a rising pressure template during expiration, thereby keeping the airway open and allow the alveoli to deflate. One example embodiment of such a respiratory pressure treatment apparatus 102 for implementing the present technology may be considered with the illustration of FIG. 6 or the PAP device 4000. In the embodiment of FIG. 6, the device includes a controller 104 to modulate treatment pressure in either or both inspiration or expiration phases in accordance with one or more control methodologies. The apparatus 102 will typically include a flow generator such as a servo-controlled blower 110. The apparatus may be configured for coupling with a patient interface, such as a delivery tube 112 and a mask 108. The mask may optionally be a nasal mask, nose & mouth mask, full-face mask or nasal pillows or other device to provide a seal with the patient's respiratory system so as to permit a pressure treatment at one or more pressures above atmospheric or ambient pressure. Optionally, the patient interface may include an endotracheal tube.

The apparatus 102 also may include sensors, such as a pressure sensor 105 and/or flow sensor 106. In such an embodiment, the pressure sensor 105, such as a pressure transducer, may measure the pressure generated by the blower 110 and generate a pressure signal p(t) indicative of the measurements of pressure, such as mask pressure or an estimate of mask pressure. Similarly, the flow sensor generates a signal representative of the patient's respiratory flow. For example, flow proximate to the patient interface or a sense tube (not shown) may be measured using a pneumotachograph and differential pressure transducer or similar device such as one employing a bundle of tubes or ducts to derive a flow signal f(t). Other sensors may be utilized to generate data indicative of flow or pressure for the purposes of the control methodologies of the apparatus.

Based on flow f(t) and pressure p(t) signals, the controller 104 with one or more processors generates blower control signals. For example, the controller may generate a desired pressure target and servo-control the blower to meet the target by comparing the target with the measured condition of the pressure sensor. Thus, the controller 104 may make controlled changes to the pressure delivered to the patient interface by the blower 110. Optionally, such changes to pressure may be implemented by controlling an exhaust with a mechanical release valve (not shown) to increase or decrease the exhaust while maintaining a relatively constant blower speed. Such changes in pressure may be based on a detection of the patient's respiratory cycle as discussed in more detail herein, such as by analysis of data from a flow signal or pressure signal of the sensors. With such a controller or processor, the apparatus can be used for many different pressure treatment therapies in accordance with a programmed pressure delivery function or equation for determining the pressure set points.

Thus, the controller 104 will typically include a processor configured to implement particular control methodologies such as the algorithms described in more detail herein. To this end, the controller may include integrated chips, a memory and/or other control instruction, data or information storage medium. For example, programmed instructions encompassing such a control methodology may be coded on integrated chips in the memory of the device. Such instructions may also or alternatively be loaded as software or firmware using an appropriate data storage medium.

### 5.2 THERAPY

Thus, in one form, the present technology comprises a method for treating a respiratory disorder by applying positive pressure to the entrance of the airways of a patient through a patient interface such as with a PAP device 4000.

### 5.3 PATIENT INTERFACE 3000

### 5.3.1 Patient Interface 3000

A traditional mask-type non-invasive patient interface 3000 is illustrated in FIG. 3a. It will typically include the following functional aspects: a seal-forming structure 3100, a plenum chamber 3200, a positioning and stabilising structure 3300, a vent 3400 and a connection port 3600 for connection to air circuit 4170. In some forms a functional aspect may be provided by one or more physical components. In some forms, one physical component may provide one or more functional aspects. In use the seal-forming structure 3100 is arranged to outwardly surround an entrance to the airways of the patient so as to facilitate the supply of air at positive pressure to the airways. In this way, an external seal is formed on the skin of the patient.

### 5.4 PAP DEVICE 4000

A PAP device 4000 in accordance with one aspect of the present technology comprises mechanical and pneumatic components 4100, electrical components 4200 and is programmed to execute one or more algorithms. The PAP device has an external housing 4010 formed in two parts, an upper portion 4012 of the external housing 4010, and a lower portion 4014 of the external housing 4010. In alternative forms, the external housing 4010 may include one or more panel(s) 4015. The PAP device 4000 comprises a chassis 4016 that supports one or more internal components of the PAP device 4000. In one form a pneumatic block 4020 is supported by, or formed as part of the chassis 4016. The PAP device 4000 may include a handle 4018.

The pneumatic path of the PAP device 4000 comprises an inlet air filter 4112, an inlet muffler 4122, a controllable pressure device 4140 capable of supplying air at positive pressure (preferably a blower 4142), and an outlet muffler 4124. One or more pressure transducer 4272 and flow transducers 4274 are included in the pneumatic path.

The pneumatic block 4020 comprises a portion of the pneumatic path that is located within the external housing 4010.

The PAP device 4000 has an electrical power supply 4210, one or more input devices 4220, a central controller, a therapy device controller 4240, a therapy device 4245, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the PAP device 4000 may include more than one PCBA 4202.

The central controller, such as controller 104, of the PAP device 4000 is programmed to execute one or more algorithm modules, including in one implementation a pre-processing module, a therapy engine module, a pressure control module, and a fault condition module.

In what follows, the PAP device 4000 is referred to interchangeably as a ventilator.

### 5.4.1 PAP Device Mechanical & Pneumatic Components 4100

### 5.4.1.1 Air Filter(s) 4110

A PAP device in accordance with one form of the present technology may include an air filter 4110, or a plurality of air filters 4110.

In one form, an inlet air filter 4112 is located at the beginning of the pneumatic path upstream of a blower 4142. See FIG. 4b.

In one form, an outlet air filter 4114, for example an antibacterial filter, is located between an outlet of the pneumatic block 4020 and a patient interface 3000. See FIG. 4b.

### 5.4.1.2 Muffler(s) 4120

In one form of the present technology, an inlet muffler 4122 is located in the pneumatic path upstream of a blower 4142. See FIG. 4b.

In one form of the present technology, an outlet muffler 4124 is located in the pneumatic path between the blower 4142 and a patient interface 3000. See FIG. 4b.

### 5.4.1.3 Pressure Device 4140

In one form of the present technology, a pressure device 4140 for producing a flow of air at positive pressure is a controllable blower 4142. For example, the blower may include a brushless DC motor 4144 with one or more impellers housed in a volute. The blower is capable of delivering a supply of air, for example about 120 litres/minute, at a positive pressure in a range from about 4 cmH₂O to about 20 cmH₂O, or in other forms up to about 30 cmH₂O.

The pressure device 4140 is under the control of the therapy device controller 4240.

### 5.4.1.4 Transducer(s) 4270

In one form of the present technology, one or more transducers 4270 are located upstream of the pressure device 4140. The one or more transducers 4270 are constructed and arranged to measure properties of the air at that point in the pneumatic path.

In one form of the present technology, one or more transducers 4270 are located downstream of the pressure device 4140, and upstream of the air circuit 4170. The one or more transducers 4270 are constructed and arranged to measure properties of the air at that point in the pneumatic path.

In one form of the present technology, one or more transducers 4270 are located proximate to the patient interface 3000.

### 5.4.1.5 Anti-Spill Back Valve 4160

In one form of the present technology, an anti-spill back valve is located between the humidifier 5000 and the pneumatic block 4020. The anti-spill back valve is constructed and arranged to reduce the risk that water will flow upstream from the humidifier 5000, for example to the motor 4144.

### 5.4.1.6 Air Circuit 4170

An air circuit 4170 in accordance with an aspect of the present technology is constructed and arranged to allow a flow of air or breathable gasses between the pneumatic block 4020 and the patient interface 3000.

### 5.4.1.7 Oxygen Delivery

In one form of the present technology, supplemental oxygen 4180 is delivered to a point in the pneumatic path.

In one form of the present technology, supplemental oxygen 4180 is delivered upstream of the pneumatic block 4020.

In one form of the present technology, supplemental oxygen 4180 is delivered to the air circuit 4170.

In one form of the present technology, supplemental oxygen 4180 is delivered to the patient interface 3000.

### 5.4.2 PAP Device Electrical Components 4200

### 5.4.2.1 Power Supply 4210

In one form of the present technology power supply 4210 is internal of the external housing 4010 of the PAP device 4000. In another form of the present technology, power supply 4210 is external of the external housing 4010 of the PAP device 4000.

In one form of the present technology power supply 4210 provides electrical power to the PAP device 4000 only. In another form of the present technology, power supply 4210 provides electrical power to both PAP device 4000 and humidifier 5000.

### 5.4.2.2 Input Devices 4220

In one form of the present technology, a PAP device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller.

In one form the input device 4220 may be constructed and arranged to allow a person to select a value and/or a menu option.

### 5.4.2.3 Central Controller or Processor 4230

In one form of the present technology, the central controller (shown as processor 4230) may be a processor suitable to control a PAP device 4000 such as an x86 INTEL processor.

A processor 4230 suitable to control a PAP device 4000 in accordance with another form of the present technology includes a processor based on ARM Cortex-M processor from ARM Holdings. For example, an STM32 series microcontroller from ST MICROELECTRONICS may be used.

Another processor 4230 suitable to control a PAP device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the processor 4230 for the PAP device 4000. For example a processor from the MSP430 family of microcontrollers, manufactured by TEXAS INSTRUMENTS, may be used.

The processor 4230 is configured to receive input signal(s) from one or more transducers 4270, and one or more input devices 4220.

The processor 4230 is configured to provide output signal(s) to one or more of an output device 4290, a therapy device controller 4240, a data communication interface 4280 and humidifier controller 5250.

The processor 4230, or multiple such processors, may be configured to implement one or more methodologies described herein such as one or more algorithms 4300 expressed as computer programs stored in memory 4260. In some cases, as previously discussed, such processor(s) may be integrated with a PAP device 4000. However, in some devices the processor(s) may be implemented discretely from the flow generation components of the PAP device, such as for purpose of performing any of the methodologies described herein without directly controlling delivery of a respiratory treatment. For example, such a processor may perform any of the methodologies described herein for purposes of determining control settings for a ventilator or other respiratory related events by analysis of stored data such as from any of the sensors described herein.

### 5.4.2.4 Clock 4232

Preferably PAP device 4000 includes a clock 4232 that is connected to processor 4230.

### 5.4.2.5 Therapy Device Controller 4240

In one form of the present technology, therapy device controller 4240 is a pressure control module 4330 that forms part of the algorithms 4300 executed by the processor 4230.

In one form of the present technology, therapy device controller 4240 is a dedicated motor control integrated circuit. For example, in one form a MC33035 brushless DC motor controller, manufactured by ONSEMI is used.

### 5.4.2.6 Protection Circuits 4250

Preferably a PAP device 4000 in accordance with the present technology comprises one or more protection circuits 4250.

One form of protection circuit 4250 in accordance with the present technology is an electrical protection circuit.

One form of protection circuit 4250 in accordance with the present technology is a temperature or pressure safety circuit.

### 5.4.2.7 Memory 4260

In accordance with one form of the present technology the PAP device 4000 includes memory 4260, preferably non-volatile memory. In some forms, memory 4260 may include battery powered static RAM. In some forms, memory 4260 may include volatile RAM.

Preferably memory 4260 is located on PCBA 4202. Memory 4260 may be in the form of EEPROM, or NAND flash.

Additionally or alternatively, PAP device 4000 includes removable form of memory 4260, for example a memory card made in accordance with the Secure Digital (SD) standard.

### 5.4.2.8 Transducers 4270

Transducers may be internal of the device, or external of the PAP device. External transducers may be located for example on or form part of the air delivery circuit, e.g. the patient interface. External transducers may be in the form of non-contact sensors such as a Doppler radar movement sensor that transmit or transfer data to the PAP device.

### 5.4.2.8.1 Flow

A flow transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In use, a signal or total flow Qt signal, from the flow transducer 4274, is received by the processor 4230. However, other sensors for producing such a flow signal or estimating flow may be implemented. For example, a mass flow sensor, such as a hot wire mass flow sensor, may be implemented to generate a flow signal in some embodiments. Optionally, flow may be estimated from one or more signals of other sensors described here, such as in accordance with any of the methodologies described in a U.S. patent application Ser. No. 12/192,247, the disclosure of which is incorporated herein by reference.

### 5.4.2.8.2 Pressure

A pressure transducer 4272 in accordance with the present technology is located in fluid communication with the pneumatic circuit. An example of a suitable pressure transducer is a sensor from the HONEYWELL ASDX series. An alternative suitable pressure transducer is a sensor from the NPA Series from GENERAL ELECTRIC.

In use, a signal from the pressure transducer 4272, is received by the processor 4230. In one form, the signal from the pressure transducer 4272 is filtered prior to being received by the processor 4230.

### 5.4.2.8.3 Motor Speed

In one form of the present technology a motor speed signal 4276 is generated. A motor speed signal 4276 is preferably provided by therapy device controller 4240. Motor speed may, for example, be generated by a speed sensor, such as a Hall effect sensor.

### 5.4.2.9 Data Communication Systems

In one preferred form of the present technology, a data communication interface 4280 is provided, and is connected to processor 4230. Data communication interface 4280 is preferably connectable to remote external communication network 4282. Data communication interface 4280 is preferably connectable to local external communication network 4284. Preferably remote external communication network 4282 is connectable to remote external device 4286. Preferably local external communication network 4284 is connectable to local external device 4288.

In one form, data communication interface 4280 is part of processor 4230. In another form, data communication interface 4280 is an integrated circuit that is separate from processor 4230.

In one form, remote external communication network 4282 is the Internet. The data communication interface 4280 may use wired communication (e.g. via Ethernet, or optical fibre) or a wireless protocol to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such remote external device 4286 may be accessible to an appropriately authorised person such as a clinician.

Preferably local external device 4288 is a personal computer, mobile phone, tablet or remote control.

### 5.4.2.10 Output Devices 4290 Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.2.10.1 Display Driver 4292

A display driver 4292 receives as an input the characters, symbols, or images intended for display on the display 4294, and converts them to commands that cause the display 4294 to display those characters, symbols, or images.

### 5.4.2.10.2 Display 4294

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.4.2.11 Therapy Device 4245

In a preferred form of the present technology, the therapy device 4245 is under the control of the control module 4330 to deliver therapy to a patient 1000 as discussed herein.

Preferably the therapy device 4245 is a positive air pressure device and/or a humidification therapy device.

### 5.5 HUMIDIFIER 5000

In one form of the present technology there is provided a humidifier 5000 comprising a water reservoir and a heating plate 5240. Such a humidifier may provide water vapor in the air.

### 5.6 PRESSURE MODULATION CONTROL

In some examples, such a controller may be configured to generate a bi-level pressure treatment such as a pressure treatment with expiratory pressure relief as described by U.S. Pat. No. 7,128,069, the entire disclosure of which is incorporated herein by reference. Thus, it may set a treatment pressure for each inspiration and may reduce the pressure by a chosen level of reduction for expiratory pressure relief (EPR). The EPR levels can make breathing more comfortable for the patient.

A typical pressure signal 200 from a pressure sensor of a bi-level pressure treatment device is illustrated in FIG. 7, which is shown on a common time scale as a flow signal 204 from a flow sensor. In some cases, such a pressure signal 200 includes an inspiratory positive airway pressure (IPAP) that is delivered, and remains generally constant during patient inspiration (shown as “INSP” in FIG. 7) and an expiratory positive airway pressure (EPAP) that is delivered, and remains generally constant, during patient expiration (shown as “EXP” in FIG. 7). Optionally, the pressure treatment may also be implemented with adjustable rise and fall times between the EPAP and IPAP, as is the specific case shown in FIG. 7. The pressure treatment may also include an adjustable cycling ratio or trigger sensitivity. There are known methods for detecting patient inspiratory phase (the initiation of which is often referred to as “triggering”) and expiratory phase (the initiation of which is often referred to as “cycling”) based on data from the sensors. Such methods may be implemented to synchronize the IPAP and EPAP pressure changes with the respiratory cycle. Additional phase detection methods may include bookkeeping phase such as that described in United States Patent Application Publication No. 2010/0101574, Fuzzy Phase such as that described in U.S. Pat. No. 6,532,957, or S-mode phase such as that described in United States Patent Application Publication No. 2011/0139153, the complete disclosures of which are incorporated herein by reference. Such trigger and cycling settings typically initiate, respectively, the beginning of an inspiratory cycle of the pressure treatment apparatus (e.g., IPAP) or the beginning of the expiratory cycle of the pressure treatment apparatus (e.g., EPAP). Small pressure perturbations near the end of the EPAP or IPAP shown in the pressure signal of FIG. 7 are attributable to pressure changes measured in the mask caused by the patient's change to inspiration from expiration or expiration to inspiration as opposed to any pressure control methodology of the bi-level controller.

However, in certain embodiments of the present technology, such as those having inspiratory pressure modulation and/or expiratory pressure modulation as discussed in the following sections, the controller may implement an algorithmic process that generates a modification of the typical bi-level pressure treatment as previously discussed.

(A) Linear Pressure Modulation

One such example methodology or algorithm of the controller 104 is illustrated in the flow chart of FIG. 8. At 332, the controller controls a respiratory pressure treatment apparatus 102 so as to generate a flow of breathable gas at the patient interface. The flow of breathable gas can be adjusted as a function of a detection of a respiratory cycle of the patient, for example, upon detection of inspiration or expiration through analysis of pressure and/or flow data from the sensors. The generated flow of breathable gas may then include an inspiratory pressure portion and an expiratory pressure portion such that the expiratory pressure portion may generally be at a pressure lower than an inspiratory pressure portion. In some cases, the generated expiratory pressure portion may include a pressure rise.

At 334, the pressure rise of the expiratory pressure portion may be controlled by the controller with a linear function of elapsed respiratory time.

For example, FIG. 9 illustrates the generated pressure under control of a linear ramp template. As illustrated in FIG. 9, the inspiratory pressure may be generated as a constant pressure such as in a typical bi-level pressure treatment. However, the expiratory pressure may be modulated to, after an initial fall, rise linearly proportional with expiratory time.

For example, upon detection of expiration, the controller may set expiratory pressure, according to a template generated by the following expiratory pressure functions:

$${P}_{exp} = [ \frac{( {T}_{cur} - {T}_{ft} )}{{T}_{l exp}} ] * A * {P}_{ps} , for {T}_{cur} \geq {T}_{ft}$$

$${P}_{exp} = [ {( 1 - \frac{{T}_{cur}}{{T}_{ft}} )}^{2} ] * B * {P}_{ps} , for {T}_{cur} < {T}_{ft}$$

Where:
Pₑₓₚ is the set point for expiratory pressure,
T_{cur} is the current sample count or time elapsed in the detected expiratory phase of respiration,
T_{ft} is a set expiratory fall time,
Tₗₑₓₚ is a set or expected expiratory duration time,
Pₚₛ is the maximum pressure support value
   and

A and B are scaling constants. These constants may be set to facilitate scaling of the expiratory pressure template. They can range across the positive real number range. One purpose of such constants could be to change the rate at which the expiratory pressure template rises. However they may be used for other purposes (e.g., to limit Pₑₓₚ to a certain range).

However, in some cases, when T_{cur}<T_{ft} a standard pressure template may be employed.

In some embodiments, the expected expiratory duration time, Tₗₑₓₚ, can be set to a constant value. Optionally, it may be a learned value based on the patient's breathing cycle. For example, if set to a constant value, it can be chosen arbitrarily to be a typical of usual patient expiratory time. Optionally, Tₗₑₓₚ may be based on some meaningful parameter relating to the detection of the patient's breathing patterns. For example, it may be some function of a previous measured inspiratory time (e.g., Tᵢ), some maximum previously measured inspiratory time (e.g., Tᵢmax), some maximum previously measured expiratory time (e.g., Tₑmax). Optionally, the expected expiratory time may be based on a detection of a FuzzyPhase, such as the phase detection variable described in U.S. Pat. No. 6,810,876, the disclosure of which is incorporated herein by reference. Optionally, Tₗₑₓₚ can be learned via applying a machine learning algorithm to the patient's breathing patterns. In one example embodiment, Tₗₑₓₚ may be calculated as an average or mean of the expiratory time (Tₑₓₚ) for a number of prior breaths (e.g., a number of breaths in a range of 2 to 7, such as 5) which may be tracked in a suitable buffer. Optionally, the scaling constant A may be calculated based on the maximum pressure support (Pps). The term “pressure support” may be considered the difference between a desired instantaneous pressure at end inspiration and the desired instantaneous pressure at end expiration. Mathematically, the computation of A may be represented as A=Pps*B, where B is gain factor which ranges between [0,1].

As illustrated in FIG. 9, based on such a linear function the generated linear expiratory rise LER occurs after a pressure drop from the IPAP level having the fall time of T_{ft}.

B. Integrated Flow Pressure Modulation

Another example methodology or algorithm that may be implemented by the controller 104 is illustrated in the flow chart of FIG. 10. At 552, the controller controls a respiratory pressure treatment apparatus 102 so as to generate a flow of breathable gas at the patient interface. The flow of breathable gas can be adjusted as a function of a detection of a respiratory cycle of the patient, for example, upon detection of inspiration or expiration through analysis of pressure and/or flow data from the sensors. The generated flow of breathable gas may then include an inspiratory pressure portion and an expiratory pressure portion such that the expiratory pressure portion may generally be at a pressure lower than an inspiratory pressure portion. In some cases, the generated expiratory pressure portion may include a pressure rise. At 554, the pressure rise of the expiratory pressure portion may be controlled by the controller as a function of the tidal volume or of a measure of the tidal volume, such as the measured exhaled flow. For example, the pressure rise may be generated by the controller as a function of a measure of the tidal volume of a plurality of prior respiratory cycles and a difference between a reference pressure and a target expiratory pressure setting. The reference pressure may, for example, include a minimum pressure for a particular period. The function of the measure of the tidal volume of the plurality of prior respiratory cycles may include any statistical evaluation, such as the calculation of a mean value, a median value, a percentile value etc.

In one such example, FIG. 11 illustrates a pressure curve controlled by implementation of an integrated flow template. In this embodiment, the inspiratory pressure may optionally be a generally constant pressure (e.g., IPAP). However, during a portion of the expiration cycle, the expiratory pressure may be modulated to rise proportionally with exhaled flow. FIG. 11 illustrates such an integrated flow expiratatory rise (shown in FIG. 11 as “IFER”) which occurs after a pressure drop from the IPAP pressure.

In some embodiments, such an integrated flow expiratory rise may be implemented by an expiratory template determined in accordance with the following process:

(a) The instantaneous tidal volume is repeatedly calculated by the following function based on data from a sensor such as data from a flow signal:

$${I}_{vt} ( i ) = ( {I}_{vt} ( i - 1 ) + \frac{\vert {Q}_{c} \vert}{{F}_{s}} )$$

where:
Iᵥₜ is the instantaneous tidal volume;
   |Q_{c}| is the absolute value of the current flow; and
Fₛ is the sampling rate:

(b) A pressure delta, P_{Δ}, may be determined as the difference between a ‘baseline’ EPAP pressure (shown as ‘BPAP’ in FIG. 11) (which is typically the minimum mask pressure during the expiration cycle) and a target EPAP (or a target pressure setting desired for the end of, or near the end of, expiration)(shown as “TPAP” in FIG. 11) according to the following:

P_{Δ}=P_{targ}−Pₑₚₐₚ

where:
Pₑₚₐₚ is the minimum or ‘baseline’ EPAP pressure; and
P_{targ} is the target EPAP.

(c) The new expiratory pressure may then be calculated according to the following:

$${P}_{exp} = ( {P}_{Δ} * A * \frac{{I}_{vt}}{{VT}_{ave}} ) + {P}_{epap}$$

where:
Pₑₓₚ is the new expiratory pressure;
Pₑₚₐₚ is a baseline or minimum EPAP pressure;
P_{Δ} is the pressure delta;
Iᵥₜ is the instantaneous tidal volume; and
A and VTₐᵥₑ are scaling constants.

The constant A may be set to facilitate scaling of the expiratory pressure template and its values range across the positive real number range. One purpose of A could be to change the rate at which the expiratory pressure template rises. However, it may be used for other purposes (e.g., Limiting Pₑₓₚ to a certain range). The scaling constant VTₐᵥₑ is calculated as an average of the patient's recent tidal volume. For example, it may be taken over a number of breaths in a range of 2 to 8, such as the average of five breaths. Averaging over a larger number of breaths is also possible and such averaging can be of the number of breaths over an entire night or treatment session. One purpose of VTₐᵥₑ is to allow scalability of Pₑₓₚ to a set IPAP.

C. Polynomial Function Templates

Another example methodology or algorithm that may be implemented by the controller 104 is illustrated in the flow chart of FIG. 12. At 772, the controller controls a respiratory pressure treatment apparatus 102 so as to generate a flow of breathable gas at the patient interface. The flow of breathable gas can be adjusted as a function of a detection of a respiratory cycle of the patient, for example, upon detection of inspiration or expiration through analysis of pressure and/or flow data from the sensors. The generated flow of breathable gas may then include an inspiratory pressure portion and an expiratory pressure portion such that the expiratory pressure portion may generally be at a pressure lower than an inspiratory pressure portion. In some cases, the generated expiratory pressure portion may include a pressure rise. At 774, the pressure rise of the expiratory pressure portion may be controlled by the controller with a polynomial function such as a quadratic function or a function of at least a degree of three. Optionally, in some cases the function may be a linear function, such as an expiratory triangular wave. Thus, the modulation of pressure during expiration may be controlled by such a function. For example, the expiratory pressure rise may be generated by the controller as a function of a cubic function, a quartic function, etc. Similarly, the modulation of pressure during inspiration may be controlled by such a function. Control methodologies for modulation of inspiratory and/or expiratory pressure by such functions may be considered in the following example embodiments.

1. Double Peak (Cubic)

FIG. 13 illustrates a double-peak template. In this embodiment, the inspiratory and expiratory pressure are both modulated based on templates derived from or with polynomial functions such as a cubic function. In this example, the inspiratory pressure is modulated during inspiration such that early cycling (initiation of expiratory phase) is achieved. As illustrated in FIG. 13, the inspiratory pressure may reach its peak level (IPAP) and fall back to the pressure level of EPAP during patient inspiration. For example, it may do so prior to about half of the inspiratory time (e.g., mid-inspiration as shown in FIG. 13 as “Mid-INSP”). Similarly, the pressure during expiration may be modulated to rise during initial expiration and fall during latter expiration. In some cases, the peak of the curve during expiration may occur prior to about half of the expiratory time (e.g., mid-expiration as shown in FIG. 13 as “Mid-EXP”).

Inspiratory Template

The following calculation can be made to derive the inspiratory pressure template for the pressure curve illustrated in FIG. 13:

Pᵢₙₛₚ=[ax³+bx²+cx+d]*Pₚₛ*A

Where:
Pᵢₙₛₚ is the new inspiratory pressure;
{a,b,c,d} are constants for the cubic function;
x is calculated based on the detection of the current phase of the patient's respiratory cycle; and
Pₚₛ is the maximum pressure support; and
A may be set to facilitate scaling of the inspiratory pressure template. It can range across the positive real number range. One purpose of such a constant could be to change the rate at which the inspiratory pressure template rises. However it may be used for other purposes (e.g., to limit Pᵢₙₛₚ to a certain range).

Mathematically x may be represented or determined according to the following conditions:

$$x = \{ \frac{{ϕ}_{curr}}{{ϕ}_{icyc}} , {ϕ}_{curr} \leq {ϕ}_{icyc} \\ \dot{x} = \{ \frac{{ϕ}_{curr} - {ϕ}_{icyc}}{{ϕ}_{i max} - {ϕ}_{icyc}} , {ϕ}_{icyc} < {ϕ}_{curr} \leq {ϕ}_{i max}$$

x={1, otherwise

where:
φ_{curr} is the current phase and, during inspiration, ranges between [1, φᵢₘₐₓ] where 1 is start of inspiration and φᵢₘₐₓ is the phase at end of inspiration;
φ_{icyc} is the phase at a point when a peak of the inspiratory pressure is achieved.

In one example embodiment, the constants of the polynomial function of the template may be set as follows:
a=−0.37759
b=−0.24482
c=1.62241
d=0

However, these constants may be adjusted to other numerical values as desired.

Expiratory Template

The following calculation may be implemented for the expiratory pressure template of the pressure curve illustrated in FIG. 13:

Pₑₓₚ=[ax³+bx²+cx+d]*Pₚₛ*A

Where:
Pₑₓₚ is the new expiratory pressure;
{a,b,c,d} are constants for the cubic function. It is noted that the above labelled constants “a”, “b”, “c” and “d”, are identically named with respect to other constants introduced in relation to the inspiratory template as well as several other sets of constants discussed herein. This naming convention should not be taken as an indication that the constants of the same or different names have the same or different values respectively. For example, in some cases they may have identical values, but in some cases they may have different values regardless of the indicated name.
x is calculated based on the current phase;
Pps is the maximum pressure support; and:
A may be set to facilitate scaling of the expiratory pressure template. It can range across the positive real number range. One purpose of such a constant could be to change the rate at which the expiratory pressure template rises. However it may be used for other purposes (e.g., to limit Pₑₓₚ to a certain range). Mathematically x may be determined according to the following conditions:

$$x = \{ \frac{{ϕ}_{ecurr}}{{ϕ}_{ecyc}} , {ϕ}_{ecurr} \leq {ϕ}_{ecyc} \\ x = \{ \frac{{ϕ}_{e max} - {ϕ}_{ecurr}}{{ϕ}_{e max} - {ϕ}_{ecyc}} , {ϕ}_{ecyc} < {ϕ}_{ecurr} \leq {ϕ}_{e max}$$

x={1,otherwise

φ_{ecurr}=φ_{curr}−φᵢₘₐₓ

where:

φ_{curr} is the current phase and, during expiration, ranges between [φᵢₘₐₓ, φₑₘₐₓ];

φᵢₘₐₓ is the phase at the start of expiration;

φₑₘₐₓ is the phase at the end of expiration;

φ_{ecyc} is a cycling point setting for an expiratory cycling point which occurs intra-expiratory;

φₑₘₐₓ can be set to a constant value or can be learned based on the patient's breathing cycle. If set to a constant value, it can be chosen arbitrarily or be based on some meaningful parameter relating to the patient's breathing patterns such as Tᵢ, Tᵢₘₐₓ, Tₑₘₐₓ or FuzzyPhase variable as previously discussed. Alternatively, φₑₘₐₓ can be a learned value by applying a machine learning algorithm to the patient's breathing patterns. In an example implementation, φₑₘₐₓ is calculated based on the time of prior breathing cycles, such as a mean duration for a plurality of prior expiratory cycles recorded in suitable buffer. For example, it may be determined based on the mean of a five breath expiratory time (Tₑₓₚ).

In an example implementation, the constants of the function may set as follows:
a=−0.37759
b=−0.24482
c=1.62241
d=0
   However, these coefficients may be set to other numerical values as desired.

2. Cubic Expiratory Pressure Rise

FIG. 14 illustrates a version of a cubic rise template. In this embodiment, the inspiratory pressure may be maintained at a generally constant pressure. At least a portion of the expiratory pressure is modulated based on a cubic function to generate the cubic expiratory rise (shown in FIG. 14 as “CER”). The following function may be implemented to control generation of such an expiratory pressure modulation.

Expiratory Template

The expiratory pressure template may be calculated with the following equation:

Pₑₓₚ=[ax³+bx²+cx+d]*Pₚₛ*A

Where:
Pₑₓₚ is the new expiratory pressure;
{a, b,c,d} are constants for the cubic function;
x is calculated based on the current phase;
Pps is the maximum pressure support; and
A may be set to facilitate scaling of the expiratory pressure template. It can range across the positive real number range. One purpose of such a constant could be to change the rate at which the expiratory pressure template rises. However it may be used for other purposes (e.g., to limit Pₑₓₚ to a certain range). In this embodiment, similar to prior embodiments, x may be determined in accordance with the following conditions:

$$x = \{ \frac{{ϕ}_{ecurr}}{{ϕ}_{e max}} , {ϕ}_{ecurr} \leq {ϕ}_{e max}$$

x={1, otherwise

φ_{ecurr}=φ_{curr}−φᵢₘₐₓ

where:
φ_{curr} ranges between [φᵢₘₐₓ, φₑₘₐₓ] in expiration;
φᵢₘₐₓ is the phase at the start of expiration; and
φₑₘₐₓ is the phase at the end of expiration. As with prior embodiments, φₑₘₐₓ may be set to a constant value or can be learned based on the patient's breathing cycle. It can be chosen arbitrarily or be based on some meaningful parameter relating to the patient's breathing patterns such as Tᵢ, Tᵢₘₐₓ, Tₑₘₐₓ or Fuzzyphase as previously mentioned. Alternatively, φₑₘₐₓ can be a learned by applying a machine learning algorithm to the patient's breathing patterns.

In an example implementation, φₑₘₐₓ may be calculated based on the time of prior breathing cycles, such as a mean, median, minimum, 95^{th} percentile, or maximum duration for a plurality of prior expiratory cycles recorded in suitable buffer. For example, it may be determined based on the mean of a five breath expiratory time (Tₑₓₚ). Optionally, it may be a mean, median, 95th percentile, max or min of a recent end expiration phase.

In an example embodiment, the constants may be set as follows:
a=0.4
b=−1.6
c=2.2
d=0

However, they can be adjusted to other numerical values as desired.

3. Cubic Whole

FIG. 15 illustrates a cubic whole template. In this embodiment, the inspiratory pressure may optionally be set to a generally constant pressure. However, the expiratory pressure is modulated based on a cubic function to include a pressure rise. Moreover, the overall pressure modulation function may include an expiratory pressure raise, followed by an expiratory pressure decline. The peak of the expiratory modulation function is often adjacent to the mid expiration point, but may be located at any point during expiration. This may be controlled with the following cubic function.

Expiratory Template

The expiratory pressure template may be set with the following equation:

Pₑₓₚ=[ax³+bx²+cx+d]*Pₚₛ*A

where:
Pₑₓₚ is the new expiratory pressure;
{a,b,c,d} are constants for the cubic function and x is calculated based on the current phase;
Pₚₛ is the maximum pressure support; and
A may be set to facilitate scaling of the expiratory pressure template. It can range across the positive real number range. One purpose of such a constant could be to change the rate at which the expiratory pressure template rises. However it may be used for other purposes (e.g., to limit Pₑₓₚ to a certain range). As previously mentioned, in this embodiment, the expiratory template is based on an expiratory cycling point which may correspond to a peak during expiration. Thus, x may be determined in accordance with the following conditions:

$$x = \{ \frac{{ϕ}_{ecurr}}{{ϕ}_{ecyc}} , {ϕ}_{ecurr} \leq {ϕ}_{ecyc} \\ x = \{ \frac{{ϕ}_{e max} - {ϕ}_{ecurr}}{{ϕ}_{e max} - {ϕ}_{ecyc}} , {ϕ}_{ecyc} < {ϕ}_{ecurr} \leq {ϕ}_{e max}$$

x=Lotherwise

φ_{ecurr}=φ_{curr}−φᵢₘₐₓ

where:
φ_{curr} ranges between [φᵢₘₐₓ, φₑₘₐₓ] in expiration;
φᵢₘₐₓ is the phase at the start of expiration;
φₑₘₐₓ is the phase at the end of expiration as previously discussed;
φ_{ecyc} is an expiratory cycling point setting for intra-expiratory cycling.

In the example implementation of FIG. 15, the coefficients are set based on the determined expiratory phase (e.g., φ_{ecurr}) with a first set selected in an early portion of expiration and a second set selected in a latter portion of expiration in conjunction with the intra-expiratory cycling point. It will be understood that different numerical values for the coefficients and intra-expiratory cycling point may be implemented as desired.

For φ_{ecurr}≦φ_{ecyc} assign the set of coefficients for expiratory pressure control as follows:
a=−0.37759
b=−0.24482
c=1.62241
d=0

For φ_{ecyc}<φ_{ecurr}≦φₑₘₐₓ assign the set of coefficients for expiratory pressure control as follows:
a=3.77759
b=1.84482
c=1.0
d=0

As illustrated in FIG. 15, the pressure generated by control of this expiratory template may include a sharp rise gradient and a sharp fall gradient. Although the above cubic functions are employed to achieve these gradients, these gradients may be achieved by other methods that mimic the expiratory pressure modulation. Moreover, variations of these gradient features may also be implemented. For example, in one such embodiment, a cubic rising function may be combined with an exponential falling function or a cubic falling function might be combined with an exponential raising function. Such a function switch may be made in accordance with the expiratory cycling point that serves as the threshold for the function adjustment.

4. Quartic Modulation

FIG. 16 illustrates a waveform generated by control of a quartic template. In this embodiment, the inspiratory pressure may optionally be a generally constant pressure. However, the expiratory pressure may be modulated according to a quartic function as follows:

Expiratory Template

The expiratory pressure template may be calculated with the following equation:

Pₑₓₚ=[ax⁴+bx³+cx²+dx+e]*Pₚₛ*A

where:
Pₑₓₚ is the new expiratory pressure;
{a, b, c, d, e} are coefficients for the quartic function;
x is calculated based on the current phase;
Pps is the maximum pressure support; and
A may be set to facilitate scaling of the expiratory pressure template. It can range across the positive real number range. One purpose of such a constant could be to change the rate at which the expiratory pressure template rises. However it may be used for other purposes (e.g., to limit Pₑₓₚ to a certain range). The variable x may be computed in accordance with the following conditions:

$$x = \{ \frac{{ϕ}_{ecurr}}{{ϕ}_{e max}} , {ϕ}_{ecurr} \leq {ϕ}_{e max}$$

x={1,otherwise

φ_{ecurr}=φ_{curr}−φᵢₘₐₓ

where:
φ_{curr} ranges between [φᵢₘₐₓ, φₑₘₐₓ] in expiration;
φᵢₘₐₓ is the phase at the start of expiration;
φₑₘₐₓ is the phase at the end of expiration, which may be set or determined as previously discussed;

The constants for the quartic function may be set to the following example default values:
a=1.0
b=−2.0
c=1
d=0
e=0

These values in conjunction with the quartic function may produce a template corresponding to the expiratory modulation shown in FIG. 11. However, these values may be adjusted to other numerical values as desired. Moreover, any suitable automated method may be used to set or adjust the constants based on different sets of coefficients. For example, the above values result in an intra-expiratory cycling point shown as the peak in mid-expiration between rise and fall gradients in FIG. 11 without an explicit expiratory cycling point setting such as φ_{ecyc}. However, in some embodiments as with the cubic function previously mentioned, such an expiratory cycling point setting φ_{ecyc} may be implemented and different coefficient sets may be selected for a given expiratory cycle based on that setting. Alternatively, in some embodiments a single set may be used for an expiratory cycle depending on the desired cycling point. For example, the function may be parameterized and solved mathematically based on the desired intra-expiratory cycling point. Thus, one or more different sets of coefficients may be implemented to permit automated shifting of the time position of the cycling point in expiration on a cycle-by-cycle basis depending on detectable patient conditions. FIG. 17 shows various pressure time curves or pressure templates with different cycle points (e.g., later or earlier than the cycle point of FIG. 16 that may be achieved by implementation of different coefficients.

FIG. 17 illustrates a family of pressure templates with quartic expiratory modulation in accordance with the example methodology of FIG. 16. The family of templates is generated by varying the quartic coefficients a, b, c, d and e, and represents a range of shapes that can be used for the pressure modulation during expiration.

Example System Architecture

A further example system architecture of a controller suitable for implementing the present pressure modulation technology is illustrated in the block diagram of FIG. 18. In the illustration, the controller 1301 for the respiratory pressure treatment apparatus (not shown) may include one or more processors 1308. The device may also include a display interface 1310 to output pressure and/or flow graphs (e.g., flow and/or pressure vs. time curves as illustrated in FIGS. 4, 6, 8, and 9-12, etc.) as described herein such as on a monitor or LCD panel. A user control/input interface 1312, for example, in the form of a keyboard, touch panel, control buttons, mouse etc., may also be provided to allow a user to activate or modify the control parameters or settings for the methodologies described herein. The device may also include a sensor or data interface 1314, such as a bus, for communicating with sensors, such as sensors 105 and 106 of FIG. 6. The data interface 1314 receives/transmits data such as programming instructions, flow data, pressure data, settings for inspiratory or expiratory pressure modulation etc. The device may also typically include memory/data storage components 1320 containing control instructions of the aforementioned methodologies (e.g., FIGS. 8-18). These may include processor control instructions for flow and/or signal processing (e.g., pre-processing methods, filters, etc.) at 1322 as discussed in more detail herein. They may also include processor control instructions for pressure control and modulation (e.g., linear functions, cubic functions, quartic functions, cycle detection, phase detection, etc.) at 1324. Finally, they may also include stored data 1326 for these methodologies such as pressure data, flow data, coefficients, coefficient sets, functions, tables, linear templates, polynomial templates, trigger settings, cycling settings, intra-inspiratory cycling settings, intra-expiratory cycling settings, other settings, etc.)

In some embodiments, the processor control instructions and data for controlling the above described methodologies may be contained in a computer readable recording medium as software for use by a general purpose computer so that the general purpose computer may serve as a specific purpose computer according to any of the methodologies discussed herein upon loading the software into the general purpose computer.

In the foregoing description and in the accompanying drawings, specific terminology, equations and drawing symbols are set forth to provide a thorough understanding of the present technology. In some instances, the terminology and symbols may imply specific details that are not required to practice the technology. For example, although the terms “first” and “second” may be used herein, unless otherwise specified, the language is not intended to provide any specified order but merely to assist in explaining distinct elements of the technology. Furthermore, although process steps in the detection methodologies have been illustrated in the figures in an order, such an ordering may not necessarily be required. Those skilled in the art will recognize that such ordering may be modified and/or aspects thereof may be conducted in parallel. Moreover, although the features described herein may be utilized independently, various combinations thereof may be made in a respiratory pressure treatment apparatus. For example, any of the expiratory pressure modulation functions described herein may be combined with any of the inspiratory pressure modulation functions described herein. Other variations can be made without departing with the spirit and scope of the technology.

### 5.7 GLOSSARY

For purposes of the present technology disclosure, in certain forms of the present technology, one or more of the following definitions may apply. In other forms of the present technology, alternative definitions may apply.

### 5.7.1 General

Air: In certain forms of the present technology, air supplied to a patient may be atmospheric air, and in other forms of the present technology atmospheric air may be supplemented with oxygen.

Continuous Positive Airway Pressure (CPAP): CPAP treatment will be taken to mean the application of a supply of air or breathable gas to the entrance to the airways at a pressure that is continuously positive with respect to atmosphere, and preferably approximately constant through a respiratory cycle of a patient. In some forms, the pressure at the entrance to the airways will vary by a few centimetres of water within a single respiratory cycle, for example being higher during inhalation and lower during exhalation. In some forms, the pressure at the entrance to the airways will be slightly higher during exhalation, and slightly lower during inhalation. In some forms, the pressure will vary between different respiratory cycles of the patient, for example being increased in response to detection of indications of partial upper airway obstruction, and decreased in the absence of indications of partial upper airway obstruction.

5.7.2 Aspects of PAP devices

Air circuit: A conduit or tube constructed and arranged in use to deliver a supply of air or breathable gas between a PAP device and a patient interface. In particular, the air circuit may be in fluid connection with the outlet of the pneumatic block and the patient interface. The air circuit may be referred to as air delivery tube. In some cases there may be separate limbs of the circuit for inhalation and exhalation. In other cases a single limb is used.

APAP: Automatic Positive Airway Pressure. Positive airway pressure that is continually adjustable between minimum and maximum limits, depending on the presence or absence of indications of SDB events.

Blower or flow generator: A device that delivers a flow of air at a pressure above ambient pressure.

Controller: A device, or portion of a device that adjusts an output based on an input. For example one form of controller has a variable that is under control—the control variable—that constitutes the input to the device. The output of the device is a function of the current value of the control variable, and a set point for the variable. A servo-ventilator may include a controller that has ventilation as an input, a target ventilation as the set point, and level of pressure support as an output. Other forms of input may be one or more of oxygen saturation (SaO₂), partial pressure of carbon dioxide (PCO₂), movement, a signal from a photoplethysmogram, and peak flow. The set point of the controller may be one or more of fixed, variable or learned. For example, the set point in a ventilator may be a long term average of the measured ventilation of a patient. Another ventilator may have a ventilation set point that changes with time. A pressure controller may be configured to control a blower or pump to deliver air at a particular pressure.

Therapy: Therapy in the present context may be one or more of positive pressure therapy, oxygen therapy, carbon dioxide therapy, control of dead space, and the administration of a drug.

Motor: A device for converting electrical energy into rotary movement of a member. In the present context the rotating member is an impeller, which rotates in place around a fixed axis so as to impart a pressure increase to air moving along the axis of rotation.

Positive Airway Pressure (PAP) device: A device for providing a supply of air at positive pressure to the airways.

Transducers: A device for converting one form of energy or signal into another. A transducer may be a sensor or detector for converting mechanical energy (such as movement) into an electrical signal. Examples of transducers include pressure sensors, flow sensors, carbon dioxide (CO₂) sensors, oxygen (O₂) sensors, effort sensors, movement sensors, noise sensors, a plethysmograph, and cameras.

Volute: The casing of the centrifugal pump that receives the air being pumped by the impeller, slowing down the flow rate of air and increasing the pressure. The cross-section of the volute increases in area towards the discharge port.

### 5.7.3 Aspects of the Respiratory Cycle

Apnea: An apnea will be said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Preferably, flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:

(i) Flattened: Having a rise followed by a relatively flat portion, followed by a fall.
(ii) Chair-shaped: Having a single local peak, the peak being at the leading edge, followed by a relatively flat portion.
(iii) Reverse-chair shaped: Having a relatively flat portion followed by single local peak, the peak being at the trailing edge.
(iv) M-shaped: Having two local peaks, one at the leading edge, and one at the trailing edge, and a relatively flat portion or a dip between the two peaks.

Hypopnea: A hypopnea will be taken to be a reduction in flow, but not a cessation of flow. In one form, a hypopnea may be said to have occurred when there is a reduction in flow below a threshold for a duration. In one form in adults, the following either of the following may be regarded as being hypopneas:

(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal flow.

Inspiratory portion of a breathing cycle: Preferably the period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow (Qpeak): The maximum value of flow during the inspiratory portion of the respiratory flow waveform.

Respiratory flow, airflow, patient airflow, respiratory airflow (Qr): These synonymous terms may be understood to refer to the PAP device's estimate of respiratory airflow, as opposed to “true respiratory flow” or “true respiratory airflow”, which is the actual respiratory flow experienced by the patient, usually expressed in litres per minute.

Tidal volume (Vt): The volume of air inhaled or exhaled during normal breathing, when extra effort is not applied.

(inhalation) Time (Ti): The duration of the inspiratory portion of the respiratory flow waveform.

(exhalation) Time (Te): The duration of the expiratory portion of the respiratory flow waveform.

(total) Time (Ttot): The total duration between the start of the inspiratory portion of one respiratory flow waveform and the start of the inspiratory portion of the following respiratory flow waveform.

Upper airway obstruction (UAO): includes both partial and total upper airway obstruction. This may be associated with a state of flow limitation, in which the level of flow increases only slightly or may even decrease as the pressure difference across the upper airway increases (Starling resistor behaviour).

Ventilation (Vent): A measure of the total amount of gas being exchanged by the patient's respiratory system, including both inspiratory and expiratory flow. When expressed as a volume per minute, this quantity is often referred to as “minute ventilation”. Minute ventilation is sometimes given simply as a volume, understood to be the volume per minute.

### 5.7.4 PAP Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow will be given the symbol Q. Total flow, Qt, is the flow of air leaving the PAP device. Vent flow, Qv, is the flow of air leaving a vent to allow washout of exhaled gases. Leak flow, Ql, is the flow rate of unintentional leak from a patient interface system. Respiratory flow, Qr, is the flow of air that is received into the patient's respiratory system.

Leak: A flow of air to the ambient. Leak may be intentional, for example to allow for the washout of exhaled CO₂. Leak may be unintentional, for example, as the result of an incomplete seal between a mask and a patient's face.

Pressure: Force per unit area. Pressure may be measured in a range of units, including cmH₂O, g-f/cm², hectopascal. 1 cmH₂O is equal to 1g-f/cm² and is approximately 0.98 hectopascal. In this specification, unless otherwise stated, pressure is given in units of cmH₂O. For nasal CPAP treatment of OSA, a reference to treatment pressure is a reference to a pressure in the range of about 4-20 cmH₂O, or about 4-30 cmH₂O. The pressure in the patient interface (or, more succinctly, mask pressure) is given the symbol Pm.

Sound Power: The energy per unit time carried by a sound wave. The sound power is proportional to the square of sound pressure multiplied by the area of the wavefront. Sound power is usually given in decibels SWL, that is, decibels relative to a reference power, normally taken as 10⁻¹² watt.

Sound Pressure: The local deviation from ambient pressure at a given time instant as a result of a sound wave travelling through a medium. Sound power is usually given in decibels SPL, that is, decibels relative to a reference power, normally taken as 20×10⁻⁶ pascal (Pa), considered the threshold of human hearing.

### 5.7.5 Terms for Ventilators

Adaptive Servo-Ventilator: A ventilator that has a changeable, rather than fixed target ventilation. The changeable target ventilation may be learned from some characteristic of the patient, for example, a respiratory characteristic of the patient.

Backup rate: a parameter of a ventilator that establishes the minimum respiration rate (typically in number of breaths per minute) that the ventilator will deliver to the patient, if not otherwise triggered.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

EPAP (or EEP): a base pressure, to which a pressure varying within the breath is added to produce the desired mask pressure which the ventilator will attempt to achieve at a given time.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the minimum value during expiration (e.g., PS=IPAP−EPAP). In some contexts pressure support means the difference which the device aims to achieve, rather than what it actually achieves.

Servo-ventilator: A ventilator that measures patient ventilation has a target ventilation, and which adjusts the level of pressure support to bring the patient ventilation towards the target ventilation.

Spontaneous/Timed (S/T)—A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

Ventilator: A mechanical device that provides pressure support to a patient to perform some or all of the work of breathing.

Ventilator inspiration and ventilator expiration: the periods during which the ventilator considers that it should deliver pressures appropriate respectively to patient inspiration and expiration. Depending on the quality of patient-ventilator synchronisation, and the presence of upper airway obstruction, these may or may not correspond to actual patient inspiration or expiration.

### 5.7.6 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.8 OTHER REMARKS

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

1. A method of control for a respiratory pressure treatment device comprising:
generating a flow of breathable gas at a patient interface, the flow of breathable gas comprising inspiratory portions and expiratory portions wherein the breathable gas during an expiratory portion is at a pressure generally lower than that during an inspiratory portion and wherein each of the expiratory portions comprises a pressure rise; and
controlling the pressure rise of the expiratory portions with a polynomial function.

2. The method of claim 1 wherein the polynomial function comprises a function of time or phase.

3. The method of any one of claim 1 or 2 wherein the polynomial function is linear.

4. The method of claim 1 wherein the polynomial function is a cubic function.

5. The method of claim 1 wherein the polynomial function is a quartic function.

6. The method of any of the preceding claims wherein the polynomial function comprises a sum of products of a set of coefficients and an input parameter, the input parameter being a measure of at least one of a respiratory flow, a respiratory phase and respiratory time.

7. The method of claim 6 wherein the set of coefficients are selected as a function of detected respiratory phase.

8. The method of claim 7 wherein a first set of coefficients is selected for an early portion of expiration and second set of coefficients is selected for a latter portion of expiration.

9. The method of any one of the preceding claims further comprising controlling a pressure decline in the expiratory portion, the pressure decline being subsequent to the pressure rise.

10. The method of claim 9 wherein the pressure decline is controlled with the polynomial function.

11. The method of claim 9 or claim 10 further wherein the control of the pressure decline is a function of an intra-expiratory cycling point setting value.

12. The method of any one of the preceding claims further comprising controlling an inspiratory portion with a polynomial function of at least a degree of three.

13. The method of claim 12 wherein the control of the pressure of the inspiratory portion decreases the pressure during patient inspiration.

14. The method of claim 13 wherein the decrease of pressure of the inspiratory portion follows an increase of pressure in the inspiratory portion.

15. The method of any one of the preceding claims wherein the controlling of the pressure rise during expiration is a further function of a maximum pressure support setting value.

16. A respiratory pressure treatment apparatus comprising:
a flow generator to generate a flow of breathable gas to a patient interface;
a sensor to measure the flow of breathable gas; and
a controller to control the flow generator to deliver a flow of breathable gas at a patient interface, the flow of breathable gas comprising inspiratory portions and expiratory portions, wherein the breathable gas during an expiratory portion is at a pressure generally lower than that during an inspiratory portion, and wherein each of the expiratory portions comprise a pressure rise;
the controller being configured to control the pressure rise of the expiratory portion with a polynomial function.

17. The apparatus of claim 16 wherein the polynomial function comprises a function of time or phase.

18. The apparatus of any one of claim 16 or 17 wherein the polynomial function is linear.

19. The apparatus of any one of claim 16 or 17 wherein the polynomial function is a cubic function.

20. The apparatus of any one of claim 16 or 17 wherein the polynomial function is a quartic function.

21. The apparatus of any one of claims 16 to 20 wherein the polynomial function comprises a sum of products of a set of coefficients and an input parameter, the input parameter being a measure of at least one of a respiratory flow, a respiratory phase and respiratory time.

22. The apparatus of claim 21 wherein the set of coefficients are selected as a function of detected respiratory phase.

23. The apparatus of claim 22 wherein a first set of coefficients is selected for an early portion of expiration and second set of coefficients is selected for a latter portion of expiration.

24. The apparatus of any one of claims 16 to 23 wherein the controller is further configured to control a pressure decline in the expiratory portion, the pressure decline being subsequent to the pressure rise.

25. The apparatus of claim 24 wherein the pressure decline is controlled with the polynomial function.

26. The apparatus of claim 24 or claim 25 further comprising an intra-expiratory cycling point setting, wherein the controller is further configured to control the pressure decline as a function of the intra-expiratory cycling point setting.

27. The apparatus of any one of claims 16 to 26 wherein the controller is further configured to control an inspiratory portion with a polynomial function of at least a degree of three.

28. The apparatus of claim 27 wherein the control of the pressure of the inspiratory portion decreases the pressure during patient inspiration.

29. The apparatus of claim 28 wherein the decrease of pressure of the inspiratory portion follows an increase of pressure in the inspiratory portion.

30. The apparatus of any, one of claims 16 to 29 wherein the control of the pressure rise is a further function of a maximum pressure support setting value.

31. A method of control for a respiratory pressure treatment device comprising:
generating a flow of breathable gas at a patient interface, the flow of breathable gas comprising inspiratory portions and expiratory portions wherein the breathable gas during an expiratory portion is at a pressure generally lower than that during an inspiratory portion and wherein the expiratory portion comprises a pressure rise; and
controlling the pressure rise of the expiratory portion with a function of a tidal volume and a difference between a baseline pressure and a target expiratory pressure setting.

32. The method of claim 31, wherein the function of a tidal volume comprises a ratio between an instantaneous tidal volume and a measure of tidal volume of prior respiratory cycles.

33. The method of claim 31 or claim 32 wherein the measure of tidal volume of prior respiratory cycles is a computed mean.

34. A respiratory pressure treatment apparatus comprising:
a flow generator to generate a flow of breathable gas to a patient interface;
a sensor to measure the flow of breathable gas; and
a controller to control the flow generator to deliver a flow of breathable gas at a patient interface, the flow of breathable gas comprising inspiratory portions and expiratory portions, wherein the breathable gas during an expiratory portion is at a pressure generally lower than that during an inspiratory portion, and wherein an expiratory portion comprises a pressure rise;
the controller being configured to control the pressure rise of the expiratory portion with a function of a tidal volume and a difference between a baseline pressure and a target expiratory pressure setting.

35. The method of claim 31, wherein the function of a tidal volume comprises a ratio between an instantaneous tidal volume and a measure of tidal volume of prior respiratory cycles.

36. The apparatus of claim 34 or claim 35 wherein the measure of tidal volume of prior respiratory cycles is a computed mean.
